(19) United States
(12) Patent Application Publication
(10) Pub. No.: US 2022/0396762 A1
(43) Pub. Date: Dec. 15, 2022

(54) GROWING PROGRAMMABLE ENZYME-FUNCTIONALIZED AND SENSE-AND-RESPONSE BACTERIAL CELLULOSE LIVING MATERIALS WITH ENGINEERED MICROBIAL CO-CULTURES

(71) Applicants: Massachusetts Institute of Technology, Cambridge, MA (US); Imperial College Innovations Limited, London (GB)

(72) Inventors: Timothy Kuan-Ta Lu, Cambridge, MA (US); Tzu-Chieh Tang, Cambridge, MA (US); Wolfgang Ott, London (GB); Tom Ellis, London (GB); Charles Gilbert, Cambridge (GB)

(73) Assignees: Massachusetts Institute of Technology, Cambridge, MA (US); Imperial College Innovations Limited, London (GB)

(21) Appl. No.: 17/636,259

(22) PCT Filed: Aug. 21, 2020
(86) PCT No.: PCT/US2020/047330

§ 371 Date: Feb. 17, 2022

Related U.S. Application Data
(60) Provisional application No. 62/889,837, filed on Aug. 21, 2019.

(51) Int. Cl. C12N 1/22; C12N 9/02

(52) U.S. Cl. CPC C12N 1/22; C12N 9/0061

(57) ABSTRACT

The disclosure provides compositions and methods for growing programmable enzyme-functionalized and sense-and-response bacterial cellulose living materials with engineered microbial co-cultures.

### RELATED APPLICATIONS

This application claims priority under 35 U.S.C. 119(e) to U.S. Provisional Application No. 62/889,837, filed Aug. 21, 2019, and entitled “Growing Programmable Enzyme-Functionalized and Sense-and-Response Bacterial Cellulose Living Materials with Engineered Microbial Co-Cultures,” which is incorporated herein by reference in its entirety for all purposes.

### TECHNICAL FIELD

The disclosure provides compositions and methods for growing programmable enzyme-functionalized and sense-and-response bacterial cellulose living materials with engineered microbial co-cultures.

### BACKGROUND

The field of engineered living materials (ELMs) aims to recreate desirable properties from natural biological materials such as, for example, self-assembly from simple raw materials, autonomous morphogenesis, diverse physical and chemical properties and the ability to sense-and-respond to environmental stimuli.

Because of its material properties, high natural yield, and genetic tractability, bacterial cellulose (BC) is an ideal natural biological material for ELM development. However, there remains a paucity of genetic tools and circuits with which to engineer BC-producing bacteria.

Accordingly, there exists a need for compositions and methods to produce bacterial cellulose living materials using engineered bacteria.

### SUMMARY

Provided herein are compositions and methods for growing programmable enzyme-functionalized and sense-and-response bacterial cellulose living materials with engineered microbial co-cultures.

Natural biological materials exhibit remarkable properties: self-assembly from simple raw materials, autonomous morphogenesis, diverse physical and chemical properties and the ability to sense-and-respond to environmental stimuli. The field of engineered living materials (ELMs) aims to recreate these properties to generate new and useful materials. Owing to its material properties, high natural yield and genetic tractability, bacterial cellulose (BC) is an ideal natural biological material for ELM development. However, there remains a paucity of genetic tools and circuits with which to engineer BC-producing bacteria. Inspired by the natural microbial community of fermented kombucha tea, the studies presented herein set out to co-culture the engineerable BC-producing bacterium Komagataeibacter rhaeticus with the model organism and synthetic biology host Saccharomyces cerevisiae. The studies provided herein first established and characterized a method for stable co-culture of K. rhaeticus and S. cerevisiae, in which the two species exhibited a symbiotic interaction. Using this system, the studies further demonstrated that S. cerevisiae can be engineered to secrete enzymes into BC, generating grown, functionalized materials. The studies presented herein further developed a method for incorporating yeast cells within the pellicle, generating living materials with tunable mechanical properties. This modular system allows for shuffling engineered S. cerevisiae strains that can sense and respond to chemical and optical inputs to be incorporated into BC, enabling a versatile biosensor platform production. As demonstrated herein, living test paper that can detect contaminants and living films that can generate images based on projected patterns has been produced. This novel and robust co-culture approach therefore empowers the sustainable growth of BC-based ELMs with programmable properties.

According to one aspect, isolated bacterial cellulose (BC)-based living compositions are provided. The BC-based living compositions include a stable co-culture of at least one bacterial cellulose (BC)-producing bacteria strain and at least one synthetic biology host organism, wherein the synthetic biology host organism has been engineered to secrete one or more enzymes into bacterial cellulose (BC), and wherein the interaction between the bacteria strain and the synthetic biology host organism in the co-culture produces a self-assembled BC-based living composition. In some embodiments, the bacteria comprises Komagataeibacter rhaeticus (K. rhaeticus). In some embodiments, the synthetic biology host organism comprises an engineered yeast strain. In some embodiments, the engineered yeast strain comprises an engineered Saccharomyces cerevisiae (S. cerevisiae) strain. In some embodiments, the BC-based living composition comprises a pellicle. In some embodiments, the pellicle comprises the BC-producing bacteria strain, the synthetic biology host organism, or both the BC-producing bacteria strain and the synthetic biology host organism.

In some embodiments, each of the one or more enzymes is linked to a cellulose binding protein or cellulose binding domain. In some embodiments, the cellulose binding protein or cellulose binding domain is CBDcex. In some embodiments, each of the one or more enzymes is linked to a secretion signal peptide. In some embodiments, expression of the one or more enzymes is controlled by a genetic circuit that responds to biological, chemical, or physical stimuli.

According to another aspect, isolated bacterial cellulose (BC)-based living compositions are provided. The compositions include a stable co-culture of Komagataeibacter rhaeticus (K. rhaeticus) and an engineered Saccharomyces cerevisiae (S. cerevisiae) strain, wherein the engineered strain of S. cerevisiae has been engineered to secrete one or more enzymes into bacterial cellulose (BC), and wherein the interaction between the K. rhaeticus and the engineered S. cerevisiae in the co-culture produces a self-assembled BC-based living composition. In some embodiments, the BC-based living composition comprises a pellicle. In some embodiments, the pellicle comprises K. rhaeticus, the engineered S. cerevisiae strain, or both K. rhaeticus and the engineered S. cerevisiae strain.

In some embodiments, each of the one or more enzymes is linked to a cellulose binding protein or cellulose binding domain. In some embodiments, the cellulose binding protein or cellulose binding domain is CBDcex. In some embodiments, each of the one or more enzymes is linked to a secretion signal peptide. In some embodiments, expression of the one or more enzymes is controlled by a genetic circuit that responds to biological, chemical, or physical stimuli.

According to another aspect, isolated engineered strains of Saccharomyces cerevisiae (S. cerevisiae) are provided. The engineered strains of S. cerevisiae secrete one or more enzymes into bacterial cellulose (BC). In some embodiments, each of the one or more enzymes is linked to a cellulose binding protein or cellulose binding domain. In some embodiments, the cellulose binding protein or cellulose binding domain is CBDcex.

In some embodiments, each of the one or more enzymes is linked to a secretion signal peptide. In some embodiments, expression of the one or more enzymes is controlled by a genetic circuit that responds to biological, chemical, or physical stimuli.

According to another aspect, isolated co-cultures of Komagataeibacter rhaeticus (K. rhaeticus) and an engineered strain of Saccharomyces cerevisiae (S. cerevisiae) are provided, wherein the engineered strain of S. cerevisiae secretes one or more enzymes into bacterial cellulose (BC). In some embodiments, each of the one or more enzymes is linked to a cellulose binding protein or cellulose binding domain. In some embodiments, the cellulose binding protein or cellulose binding domain is CBDcex.

In some embodiments, each of the one or more enzymes is linked to a secretion signal peptide. In some embodiments, expression of the one or more enzymes is controlled by a genetic circuit that responds to biological, chemical, or physical stimuli.

According to another aspect, methods of producing a bacterial cellulose (BC)-based living composition are provided. The methods include creating a stable co-culture of at least one bacterial cellulose (BC)-producing bacteria strain and at least one synthetic biology host organism, wherein the synthetic biology host organism strain has been engineered to secrete one or more enzymes into bacterial cellulose (BC), and wherein the interaction between the bacteria strain and the synthetic biology host organism in the co-culture produces a self-assembled BC-based living composition. In some embodiments, the synthetic biology host organism comprises an engineered yeast strain. In some embodiments, the engineered yeast strain comprises an engineered S. cerevisiae strain. In some embodiments, the bacterial strain comprises K. rhaeticus. In some embodiments, each of the one or more enzymes is linked to a cellulose binding protein or cellulose binding domain. In some embodiments, the cellulose binding protein or cellulose binding domain is CBDcex.

In some embodiments, each of the one or more enzymes is linked to a secretion signal peptide. In some embodiments, expression of the one or more enzymes is controlled by a genetic circuit that responds to biological, chemical, or physical stimuli.

In some embodiments, the bacteria strain and the synthetic biology host organism are co-cultured for at least about 3 days. In some embodiments, the bacteria strain and the synthetic biology host organism are co-cultured in a culture medium that has a higher density than the bacteria strain and the synthetic biology host organism. In some embodiments, the bacteria strain and the synthetic biology host organism are co-cultured in sucrose-containing media.

According to another aspect, methods of producing a bacterial cellulose (BC)-based living composition are provided. The methods include creating a stable co-culture of Komagataeibacter rhaeticus (K. rhaeticus) and an engineered Saccharomyces cerevisiae (S. cerevisiae) strain, wherein the engineered strain of S. cerevisiae has been engineered to secrete one or more enzymes into bacterial cellulose (BC), and wherein the interaction between the K. rhaeticus and the engineered S. cerevisiae in the co-culture produces a self-assembled BC-based living composition. In some embodiments, each of the one or more enzymes is linked to a cellulose binding protein or cellulose binding domain. In some embodiments, the cellulose binding protein or cellulose binding domain is CBDcex.

In some embodiments, each of the one or more enzymes is linked to a secretion signal peptide. In some embodiments, expression of the one or more enzymes is controlled by a genetic circuit that responds to biological, chemical, or physical stimuli.

In some embodiments, the K. rhaeticus and the engineered S. cerevisiae are co-cultured for at least about 3 days. In some embodiments, the K. rhaeticus and the engineered S. cerevisiae are co-cultured in a culture medium that has a higher density than the K. rhaeticus and the engineered S. cerevisiae. In some embodiments, the bacteria strain and the synthetic biology host organism are co-cultured in sucrose-containing media.

In some embodiments, the BC-based living composition comprises an engineered living material (ELM). In some embodiments, the BC-based living composition comprises a pellicle.

According to another aspect, uses of the isolated bacterial cellulose (BC)-based living compositions as a biosensor are provided.

According to another aspect, uses of the isolated BC-based living compositions for detecting microbe-microbe interactions are provided.

According to another aspect, uses of the isolated BC-based living composition in a method for the detection and/or degradation of an environmental pollutant are provided.

In some embodiments, the environmental pollutant is one or more β-lactam antibiotics, one or more estrogen hormones, or a combination thereof.

According to another aspect, uses of the isolated BC-based living composition for detecting one or more pathogens in a sample are provided.

According to another aspect, uses of the isolated BC-based living composition for detecting one or more biomarkers in a sample are provided.

According to another aspect, uses of the isolated BC-based living composition in a living test paper or living film are provided. In some embodiments, the living test paper or living film generates an image in response to one or more stimuli. In some embodiments, the one or more stimuli is light.

These and other aspects of the disclosure, as well as various embodiments thereof, will become more apparent in reference to the drawings and detailed description of the disclosure.

### BRIEF DESCRIPTION OF THE DRAWINGS

FIGS. 1A-1E are a series of graphs and illustrations depicting generating co-cultures of engineerable S. cerevisiae and K. rhaeticus. FIG. 1A: An image of a BC pellicle, a flexible but tough material. FIG. 1B: Home-brewed kombucha tea. Both a large submerged mass of BC from previous rounds of fermentation as well as a newly-formed thinner layer at the surface can be seen. FIG. 1C: Schematic of the aim, to co-culture BC-producing bacteria with engineerable S. cerevisiae yeast. FIG. 1D: Images of mono-cultures and co-cultures grown for 3 days. S. cerevisiae grows well in both YPD and YPS media, forming a sediment at the base of the culture. K. rhaeticus grew well in YPD medium, forming a thick pellicle layer at the air-water interface, but failed to form a pellicle in YPS medium. When co-cultured, in both YPD and YPS, a thick pellicle layer was formed as well as a sediment layer at the base of the culture, indicating both S. cerevisiae and K. rhaeticus had grown. The left panel shows a top view from the different cultures in a 24-well plate. The right panel shows a side view from the different cultures incubated in 20 ml reaction tubes. Cell counts of K. rhaeticus. FIG. 1E: Cell counts of K. rhaeticus and S. cerevisiae were determined by plating and counting the numbers of cells present in the two phases of co-cultures—the liquid layer and the pellicle layer. Cell counts were determined for K. rhaeticus grown in mono-culture in YPD (Kr YPD) or YPS (Kr YPS) or in co-culture with S. cerevisiae in YPS (Co YPS). Cell counts were determined for S. cerevisiae grown in mono-culture in YPS (Sc YPS) or in co-culture with K. rhaeticus in YPS (Co YPS). Samples prepared in triplicate, data represent the mean±1 SD.

FIGS. 2A-2L are a series of schematic representations, graphs, and images depicting that engineered co-cultures can produce enzyme-functionalized BC materials. FIG. 2A: Schematic illustrating the concept of functionalization. S. cerevisiae cells, the majority of which are found in the liquid layer, secrete a protein which then becomes incorporated into the BC layer, conferring a new functional property to the material. FIG. 2B: BLA-secreting strains yCG04 (BLA) and yCG05 (BLA-CBD). FIG. 2C: Nitrocefin is converted from a yellow substrate to a red product in the presence of β-lactamase enzyme. FIG. 2D: The nitrocefin assay, was performed with cut, native wet pellicle samples FIG. 2E and with dried, then re-hydrated pellicle samples FIG. 2F from co-cultures with S. cerevisiae BY4741 (WT), yCG04 (BLA) and yCG05 (BLA-CBD). Images are shown of pellicles after indicated time points during the assay. In addition, the yellow-to-red color change was then quantified as green channel intensity using ImageJ image analysis software. Samples prepared in triplicate, data represent the mean±1 SD. FIG. 2F: Absolute β-lactamase activities were calculated from native, hydrated pellicles (wet) and from pellicles dried, then re-hydrated after the indicated time periods. Samples presented here are from pellicles grown in co-culture with yCGO5 (BLA-CBD). Since pellicle liquid volume is altered by drying, β-lactamase activity is represented by enzyme activity units per unit of pellicle area, to enable cross-comparison.

FIG. 2G: SEM image of S. cerevisiae from which the Mel1 enzyme was cloned. FIG. 2H: X-α-gal is converted from a colorless substrate to a blue product by Mel1 enzyme. FIG. 2L The X-α-gal assay was performed with wet and dried, then re-hydrated pellicle samples from co-cultures with the GFP-secreting strain yCGO1 (-ive) or a strain engineered to secrete Mel1 (yCG21), samples prepared in triplicate. FIG. 2J: Image of the fungus C. troggi from which the CtLcc1 enzyme was cloned. FIG. 2K: ABTS is converted from a colorless substrate to a green product by laccase enzyme. FIG. 2L: The ABTS assay was performed with wet and dried, then re-hydrated pellicle samples from co-cultures with the GFP-secreting strain yCGO1 (-ive) or a strain engineered to secrete CtLcc1 (yCG18), samples prepared in triplicate.

FIGS. 3A-3F are a series of schematic representations, graphs, and images depicting incorporation of yeast cells within BC material. FIG. 3A: Schematic demonstrating that modified medium density can facilitate the incorporation of yeast cells in the pellicle.

FIG. 3B: Co-cultures were prepared in YPS media with or without 45% OptiPrep. Images show the pellicles formed at the air-water interface and the liquid below the pellicle, following pellicle removal. FIG. 3C: Isolated pellicles from YPS and YPS+OptiPrep co-cultures. Pellicles isolated from co-cultures with OptiPrep have a speckled appearance, due to the presence of S. cerevisiae cells. FIG. 3D: Yeast colony forming unit (CFU) counts from pellicles grown in YPS and YPS+OptiPrep. Data represent the mean and SD. *** p<0.001.

FIG. 3E: BET surface area of the pellicles. Data represent the mean and SD. ** p<0.01. FIG. 3F: SEM images of the bottom surface and cross section of pellicles. The image of bottom surface of pellicle in YPS+OptiPrep is magnified from the red dash-line circle shown in the bottom right corner of the top left panel of FIG. 3F.

FIGS. 4A-4I are a series of schematic representations and graphs depicting modifying physical properties of BC materials. FIG. 4A: Schematic showing the yCelMix cells secreting cellulases into surrounding microenvironment from the bottom surface of the pellicle. FIG. 4B: Schematic illustrating the architecture of yCelMix cellulase secretion strain. Expression of each cellulase is controlled by distinct combination of promoter (a strong promoter selected from the yeast toolkit (YTK) system²⁷) and terminator to prevent potential homologous recombination. CBH1 (cellobiohydrolase from Chaetomium thermophilum), CBH2 (cellobiohydrolase from Chrysosporium lucknowense), BGL1 (β-glucosidase from Saccharomycopsis fibuligera), and EGL2 (endoglucanase from Trichoderma reesei) are fused with their optimal secretion signals as described in Lee et al while LPMO (LPMO9H from Podospora anserine) was fused with S. cerevisiae MFα signal peptide. Secretion signal peptides are colored in grey (NS: native signal sequence; TFP13: translational fusion partner 13; TFP19: translational fusion partner 19). Cleavage sites for the cellulases on cellulose polymer chain are marked by red dash line circles. Each circle represents a monosaccharide unit in the cellulose polymer chain. FIG. 4C: Normalized pellicle dried weight of cellulase secreting yeast plus WT K. rhaeticus. Red dash line represents 100% of wild type dried weight. FIG. 4D: Stress-strain curves of dried WT (BY4741) and yCelMix pellicles. Samples are from 7 WT and 6 yCelMix co-cultures. FIG. 4(e-g): Tensile strength at break, strain at break, and Young's modulus calculated from the data in FIG. 4D. Data represent the mean and SD. *** p<0.001. FIG. 4(h, i): Rheological properties of WT and yCelMix pellicles measured by FIG. 4H strain sweep at 1 rad/s and FIG. 4I frequency sweep at 1% strain. Arrows in FIG. 4I indicate the crossover of the storage modulus (G′, elastic deformation) and loss modulus (G″, viscous deformation).

FIGS. 5A-5J are a series of schematic representations and images depicting that engineered co-cultures can produce living BC sense-and-response materials. FIG. 5A: Schematic illustrating sense-and-response pellicle function. Pellicles are grown, containing engineered S. cerevisiae strains capable of detecting environmental stimuli and responding by adapting the material properties in some way. FIG. 5B: Schematic showing genetic GPY093 circuit. The engineered S. cerevisiae strain (GPY093) senses the presence of the chemical inducer β-estradiol (BED) and in response produces the reporter protein GFP. The Z3EV synthetic transcription factor is expressed from the weak constitutive yeast promoter pREV1. Upon addition of β-estradiol Z3EV is able to bind Z3EV binding sites (Z3BSs) in the pGAL1 promoter, activating transcription of GFP. FIG. 5C: Testing biosensor pellicles. Pellicles with either BY4741 (WT) or β-estradiol (BED) responsive (GPY093) S. cerevisiae incorporated within the BC matric were grown using the OptiPrep method. Triplicate samples were washed, then incubated with agitation in fresh media in the presence or absence of BED. After 24 hours, pellicles were washed and imaged for GFP fluorescence under a transluminator. FIG. 5D: Pellicles into which S. cerevisiae cells have been incorporated can be dried and stored. FIG. 5E: Schematic illustrating the dried pellicle biosensor experimental set up. Dried pellicles into which biosensor S. cerevisiae are incorporated are incubated in fresh medium with or without the inducer and later screened for response. Dried pellicles, into which GPY093 was incorporated, were incubated in fresh medium without agitation in the presence or absence of BED following storage for 1 day FIG. 5F or 4 months FIG. 5G. After 24 hours, pellicles were imaged for GFP fluorescence under a transluminator. Samples prepared in triplicate. FIG. 5H: Dried pellicles, into which a GPCR-based MFα-responsive S. cerevisiae strain (yWS890) was incorporated, were incubated in fresh medium without agitation in the presence or absence of MFα. After 24 hours, biosensor pellicles were imaged for GFP fluorescence under a transluminator. Samples prepared in triplicate. FIG. 5L Schematic illustrating the yCG23 construct design and function. Similarly to GPY093, yCG23 enables BED-inducible expression and secretion of the CtLcc1 laccase. FIG. 5J: Native, wet pellicles from YPS-OptiPrep co-cultures of the GFP-secreting yCGO1 strain (WT) and yCG23 were harvested, washed and inoculated into fresh medium with or without BED. After 24 hours growth, pellicles were again harvested and washed and assayed for laccase activity using the colorimetric ABTS assay. Samples prepared in triplicate.

FIGS. 6A-6E are a series of schematic representations and images depicting optical patterning of enzymatically functionalized BC materials. FIG. 6A: Schematic showing the optogenetic circuit. The engineered S. cerevisiae strain (yNC and yNS) senses the presence of blue light and in response produces the reporter protein NanoLuc. The LexA-CRY2 and VP16-CIB synthetic transcription factors are expressed from the weak constitutive yeast promoter pREV1 and strong constitutive yeast promoter pTDH3, respectively. Upon exposure to light, the dimer is able to bind LexA binding sites (8×LexA-op) in the pLEXA promoter, activating transcription of NanoLuc. FIG. 6B: Schematic illustrating the two modes of functionalization. The yNC strain secrete NanoLuc-CBD which diffuse into and eventually binds to the surrounding cellulose matrix, while the yNS strain display NanoLuc-SED1 on the cell surface. FIG. 6C: The pellicles grown in dark or light after 3 days. FIG. 6D: yNC and yNS pellicles were grown in dark and then exposed to light under a mask for 4 hours. NanoLuc substrate was applied in the end for visualization of the pattern created by masking. FIG. 6E: Same pellicles grown in dark were subjected to a complicated pattern created by a projector.

FIGS. 7A-7D are a series of images depicting images of cultures and pellicles from the co-culture condition screen. S. cerevisiae (Sc) and K. rhaeticus (Kr) were inoculated in mono-culture or co-culture (Co) in rich yeast media (YEP) or BC-producing bacteria media (HS) with either glucose or sucrose as the carbon source. For co-cultures, the Sc pre-cultures were diluted into fresh medium over a range from 1/100 (Sc 10⁻²) to 1/10⁶ (Sc 10⁻⁶). In mono-culture, Sc pre-cultures was diluted 1/100 and Kr pre-cultures was diluted 1/50. As a control for contamination, wells were included in which no cells were inoculated (BLANK). After 4 days incubation at 30° C., images were taken of cultures and then of isolated pellicle layers, where present. Cultures (FIG. 7A) and pellicles (FIG. 7B) produced in HS media and cultures (FIG. 7C) and pellicles (FIG. 7D) produced in YEP media.

FIG. 8 is a schematic representation depicting the process of defining and testing a standard protocol for co-culturing S. cerevisiae and K. rhaeticus. Schematic outlining the standard co-culture protocol. K. rhaeticus and S. cerevisiae are grown in mono-culture under agitation. K. rhaeticus cultures are then centrifuged and resuspended in YEP-sucrose (YPS) medium to a final OD₆₀₀=2.5—this step removes trace amounts of cellulase enzyme and normalizes cell density. S. cerevisiae cultures are normalized by diluting to an OD₆₀₀=0.01 in YPS. Normalized K. rhaeticus and S. cerevisiae cultures are then inoculated into fresh YPS by diluting 1/50 and 1/100, respectively.

FIG. 9 is a graph depicting measuring co-culture pellicle yields. To follow BC production dynamics over time, co-cultures were prepared following this standard protocol and left to incubated over several days. At each time point, pellicle layers were removed and dried, since pellicles consist of ˜99% water. Once dried, pellicles were weighed to determine the pellicle yield. Since pellicles were not treated to lyse and remove cells, this measurement includes contribution from both BC yield and entrapped cells. Pellicle yield rapidly increased between 2 and 3 days, at which point it plateaus. Samples prepared in triplicate, data represent the mean±1 SD.

FIG. 10 is a series of images depicting fluorescence microscopy images of pellicles. Three separate regions of the same pellicle are shown, illustrating the variability in the density of GFP-expressing S. cerevisiae cells within the pellicle. Brightfield and GFP images were taken using a 20× objective and merged.

FIGS. 11A-11C are a series of schematic representations and images depicting the process of investigating co-culture stability by passage. FIG. 11A: Co-cultures of S. cerevisiae Sc GFP and K. rhaeticus Kr RFP were passaged by iteratively back-diluting liquid from below the pellicle layer in mature co-cultures into fresh YPS medium. FIG. 11B: At each stage, mature pellicles were imaged. Pellicle formation was constant, indicating K. rhaeticus was growing well. In addition, a clear sediment was formed below the pellicle, consistent with S. cerevisiae growth. FIG. 11C: To confirm the presence of the initial S. cerevisiae strain, which expresses GFP, in passage co-cultures, samples of the liquid below the pellicle (LIQUID) and enzymatically-degraded pellicles (PELLICLE) were plated and imaged for GFP fluorescence. In the interest of clarity, plates from only three time points are shown here. The appearance of the final time point is different as it was imaged for fluorescence using different equipment (fluorescence scanning versus imaging under a transluminator). The images showed that the initial GFP-expressing S. cerevisiae strain was maintained throughout passage.

FIGS. 12A-12C are a series of schematic representations, graphs, and images depicting a putative mechanism for S. cerevisiae stimulation of K. rhaeticus growth. FIG. 12A: Various studies report that yeast (green pear-shaped objects) in kombucha microbial communities degrade extracellular sucrose to glucose and fructose which both yeast and BC-producing bacteria (red oblong rod-shaped objects) consume to produce biomass. FIG. 12B: Pellicle yields were compared between K. rhaeticus mono-culture and co-culture. Since K. rhaeticus grows poorly in sucrose media, mono-culture was performed in YPD (Kr YPD) and compared to co-culture in YPS (Co YPS). Co-culture was found to result in only a slight, but significant decrease in pellicle yield. Samples prepared in triplicate, p<0.01 from unpaired, two-tailed t-test, data represent the mean±1 SD. FIG. 12C: A variety of cultures were prepared in YPS: K. rhaeticus Kr RFP mono-culture (Kr YPS), co-cultures of Kr RFP and S. cerevisiae yWS195 (co-culture YPS) and mono-cultures of K. rhaeticus Kr RFP spiked with a range of dilutions of a stock solution of commercial S. cerevisiae invertase at 5000 U/mL concentration. Images were taken of cultures and, where present, isolated pellicles after 3 days of incubation at 30° C.

FIGS. 13A-13F are a series of graphs and schematic representations depicting the reproducibility of co-culture pellicle yields and cell densities. FIG. 13A: Pellicle yields were measured on three separate occasions. For each repeat samples were prepared in triplicate, horizontal bars represent the mean±1 SD, green circles represent the values of individual samples. FIG. 13B: Cell counts from co-cultures were prepared on three separate occasions by plating onto selective media and scanning for RFP fluorescence for K. rhaeticus and GFP fluorescence for S. cerevisiae. Cell counts were recorded from both the liquid and pellicle layers for both K. rhaeticus (FIG. 13C) and S. cerevisiae (FIG. 13D). Samples were prepared in triplicate, p-values calculated by unpaired, two-tailed t-test data (* p<0.05 and ** p<0.005), data represent the mean±1 SD. Since logarithmic scales can mask some of the variation, numerical values of cell counts are included for both K. rhaeticus (FIG. 13E) and S. cerevisiae (FIG. 13F).

FIG. 14 is a graph depicting secreted β-lactamase activity in S. cerevisiae mono-culture. Culture supernatants from WT, BLA and BLA-CBD strains were assayed for β-lactamase activity using the colorimetric nitrocefin substrate. The product formation rate was measured using a plate reader. Samples prepared in triplicate, data represent the mean±1 SD.

FIGS. 15A-15B are a series of images depicting wet and dried BC pellicles. FIG. 15A: A piece of BC pellicle in its native, wet stated. FIG. 15B: A piece of a BC pellicle following drying using the sandwich method. Dried pellicles are much thinner than wet pellicles due to water loss and are similar in appearance to thin sheets of paper.

FIGS. 16A-16D are a series of graphs depicting BLA assay standard curves. To calculate absolute β-lactamase activities, standard curves were run alongside wet (FIG. 16A), dry 0 d (FIG. 16B), dry 1 m (FIG. 16C) and dry 6 m (FIG. 16D) samples.

FIG. 17 is a graph depicting retention of β-lactamase within functionalized material after washing. Dried pellicles functionalized with BLA or BLA-CBD were subjected to a variable number of washes in PBS buffer and then screened for β-lactamase activity by the nitrocefin assay. Samples prepared in triplicate, data represent the mean±1 SD.

FIGS. 18A-18C are a series of schematic representations and images depicting secretion of the alpha-galactosidase Mel1. FIG. 18A: Two engineered strains were generated for Mel1 secretion. The first possessed the Mel1 N-terminal signal peptide and Mel1 catalytic region fused to CBDcex (yCG20). The second possessed the MFα signal peptide fused to the Mel1 catalytic region and CBDcex (yCG21). In both constructs, expression was driven by the strong constitutive promoter pTDH3. FIG. 18B: Strains were screened for Mel1 secretion by a plate-based colorimetric assay. Transformants were re-streaked in triplicate on SC URA⁻ agar supplemented with the colorimetric reporter X-α-gal. After two days growth activity was detectable in the form of halos of blue pigment around colonies of both yCG20 and yCG21. No blue pigment was formed around colonies of the negative control strain, GFP-secreting yCGO1 (-ive). Since the growth rate of yCG20 was severely reduced compared to that of yCG21 and yCG01, yCG21 was taken forwards for BC material functionalization. FIG. 18C: X-α-gal is a colorimetric reporter for Mel1; in the presence of active α-galactosidase enzymes, X-α-gal is converted from a colorless substrate to a blue pigment.

FIGS. 19A-19C are a series of schematic representations and images depicting secretion of fungal laccase enzymes. FIG. 19A: Four engineered strains were constructed for laccase enzyme secretion. Two strains were engineered to secrete a laccase from Myceliophthora thermophila (MtLcc1) with either the native signal peptide (yCG16) or the MFα signal peptide (yCG17). Two strains were engineered to secrete a laccase from Coriolopsis trogii (CtLcc1) with either the native signal peptide (yCG18) or the MFα signal peptide (yCG19). The constructs possessed a C-terminal CBD fusion and were expressed from the strong constitutive promoter pTDH3. FIG. 19B: Strains were screened for laccase secretion by a plate-based colorimetric assay. Transformants were re-streaked on SC URA⁻ agar supplemented with the colorimetric reporter 2,2′-azino-bis(3-ethylbenzothiazoline-6-sulphonic acid) (ABTS) and CuSO₄. After two days growth, activity was detectable in the form of halos of green pigment around colonies of only yCG18. By contrast, no green pigment was formed around colonies of the negative control strain, GFP-secreting yCGO1 (-ive) nor yCG16, yCG17 or yCG19 (although low level activity could be detected after longer incubation times). Therefore, yCG18 was taken forwards for BC material functionalization. FIG. 19C: ABTS is a colorimetric reporter for laccase activity; in the presence of active laccase enzyme, ABTS is converted from a colorless substrate to a green pigment.

FIGS. 20A-20D are a series of schematic representations and images depicting the testing for functionalization of BC by GFP secretion. Culture supernatants (FIG. 20A) of S. cerevisiae strains were prepared to isolate secreted protein fraction. FIG. 20B: Supernatants from three S. cerevisiae strains were imaged for GFP fluorescence under a transluminator: yCGO4 (-ive), yCGO1 (GFP) and yCGO2 (GFP-CBD). To test if GFP-secreting strains could functionalize pellicles, co-cultures were prepared with BY4741 (WT), yCGO1 (GFP) and yCGO2 (GFP-CBD) S. cerevisiae strains and grown for 7 days (FIG. 20C) or 14 days (FIG. 20D) and imaged for GFP fluorescence with a fluorescence laser scanner. Other than mild fluctuations, likely due to slight differences in pellicle thickness, no difference in GFP signal was observed between samples.

FIGS. 21A-21D are a series of schematic representations, images, and graphs depicting cell viability in dried pellicles. FIG. 20A: Schematic illustrating the effect of optiprep in the culture medium. By increasing culture medium density, S. cerevisiae cells become buoyant, rise to the surface and become incorporated into the BC matrix. FIG. 20B: Pellicles into which S. cerevisiae cells have been incorporated can be dried and stored. FIG. 20C: Cell viability was compared between wet and dried pellicles by enzymatically-degraded pellicles, plating and obtain counts of fluorescent S. cerevisiae cells. Bars represent the mean and green dots represent individual values. FIG. 20D: After storage for 1 month, dried pellicles were enzymatically-degraded and 100 μL samples plated without dilution. The resultant plates are shown here, imaged for GFP fluorescence under translumination. Viable cells were obtained on two of three plates, indicating that a small minority of S. cerevisiae cells survive even after 1 month of storage at room temperature.

FIGS. 22A-22B are a series of schematic representations and images depicting BED-inducible CtLcc1 laccase secretion. FIG. 22A: A strain, yCG23, was constructed for engineered CtLcc1 secretion in response to the presence of β-estradiol (BED). A two-gene construct was assembled using the YTK cloning system. The first gene encoded constitutive weak expression of the BED-responsive synthetic transcription factor Z3EV. The second gene encoded a fusion of the CtLcc1 signal peptide and catalytic region fused to a C-terminal CBDcex domain, all under control of the Z3EV-responsive promoter. In presence of BED, Z3EV will translocate to the nucleus, bind the Z3 binding sites (Z3BS) upstream of the CtLcc1-CBD ORF and induce transcription. FIG. 22B: Transformants were re-streaked in triplicate onto agar containing the colorimetric reporter of laccase activity, ABTS and CuSO₄ in the presence or absence of BED. After three days of growth, a clear halo of green pigment was observed only in the presence of BED, indicating successful induction of CtLcc1 secretion.

FIGS. 23A-23C are a series of images depicting additional SEM images of dried pellicles grown in YPS+OptiPrep. FIG. 23A: Top surface of the pellicle. FIG. 23B: Bottom surface of the pellicle. Spherical yeast cells are trapped in the cellulose matrix produced by rod shape K. rhaeticus. FIG. 23C: Cross section of the pellicle. Part of the bottom surface is presented on the left.

FIGS. 24A-24B are a series of graphs depicting the yeast cell count and pellicle dry weight across 10 passages. FIG. 24A: Yeast colony forming unit (CFU) of pellicle from the first passage to the tenth passage. Each passage was inoculated using liquid from the previous passage. Data represent the mean±1 SD from biological triplicates. FIG. 24B: Pellicle dry weight from the first passage to the tenth passage. Pellicles were freeze-dried using a lyophilizer. Data represent the mean±1 SD from biological triplicates.

FIG. 25 is a graph depicting the total cellulase activity of yCelMix. The total cellulase activity of yCelMix saturated culture was calculated from standards prepared with T. reesi cellulase mix. Data represent the mean from biological triplicates.

FIG. 26 is a graph depicting that BET surface area is increased in yCelMix pellicle. Total BET surface area was calculated from dried pellicles. Data represent the mean±1 SD from biological triplicates. ** p<0.01.

FIGS. 27A-27B are a series of schematic representations and graphs depicting single cellulase secretion strains and their effect on BC stiffness. FIG. 27A: Schematic illustrating the construction of single cellulase secretion strains. XTH3 is an Arabidopsis thaliana cell-wall enzyme which catalyzes covalent cross-linking between cellulose. Each one of them is driven by a strong constitutive promoter, pTDH3. FIG. 27B: Young's moduli of dried pellicles from single cellulase secretion strains and a cross-linking enzyme secretion strain. Data represent the mean±1 SD from biological triplicates.

FIGS. 28A-28B are a series of schematic representations and graphs depicting the optimization of light induction. FIG. 28A: Schematic illustrating the design of 4 combinations of promoter strength pairs. The strong constitutive promoter (pTDH3) is marked in red and labeled as pTDH3 and the weak constitutive promoter (pREV1) is marked in blue and labeled as pREV1. They drive the expression of the DNA-binding component and activation component, which together drive the expression of GFP in the presence of light. FIG. 28B: GFP expression of yeast culture in liquid in dark or after 4 hours of light induction. Data represent the mean±1 SD from biological triplicates.

FIGS. 29A-29C are a series of graphs and images depicting light induction of luciferase expression and the tunable resolution on BC living films. FIG. 29A: Bioluminescence of yNC and yNS liquid culture after 4 hours of light induction. Data represent the mean±1 SD from biological triplicates. FIG. 29B: yNS ASED1 pellicle after 12 hours of exposure to projected pattern. This knockout strain showed slower growth compared to yNS. Less foci were formed and they are unable to provide enough resolution to reflect the pattern. FIG. 29C: yNC pellicle after 1-day outgrowth followed by 12 hours exposure. Increased yeast cell number on the surface provides higher resolution for patterning. Higher background activity reflects the diffusion of NanoLuc-CBD within the BC material.

### DETAILED DESCRIPTION

Provided herein are compositions and methods for growing programmable enzyme-functionalized and sense-and-response bacterial cellulose living materials with engineered microbial co-cultures.

Living organisms produce materials with remarkable properties. As cells grow and divide, they can synthesize a range of complex biopolymers while simultaneously controlling the patterning and morphogenesis of the resultant materials with incredible precision and over multiple length scales. This basic process of biological material assembly, typically fed by simple raw materials and occurring under mild conditions, enables the production of materials with a vast range of functional properties. Further, living cells within biological materials are poised to sense and respond to changes in their environment, allowing dynamic remodeling of material properties in response to defined chemical and physical stimuli.

The field of engineered living materials (ELMs) aims to take advantage of these properties by genetically-programming cells to assemble novel, useful materials¹⁻⁴. However, rationally engineering cells to produce materials with the complexity of natural biological materials remains a major challenge. Numerous recent reports have described engineering biological material assembly in simple, genetically-tractable microbial systems. In particular, Escherichia coli biofilm nanomaterials have attracted great interest as model ELM systems. Curli fibers, amyloid polymers of the secreted CsgA protein, are a major component of the extracellular structural matrix of E. coli biofilms⁵. By engineering the CsgA monomer and controlling its expression, biofilm ELMs with electrical conductivity⁶⁻⁸, autonomous patterning⁹, metal adhesion¹⁰, catalytic activity^{11,12} and environmental sense-and-response functions¹³ have been created. However, production scalability remains a major limitation to potential applications of engineered curli nanomaterials; even under optimized conditions, the yields of E. coli biofilm ELMs remain restricted to tens of milligrams per liter of culture¹⁴.

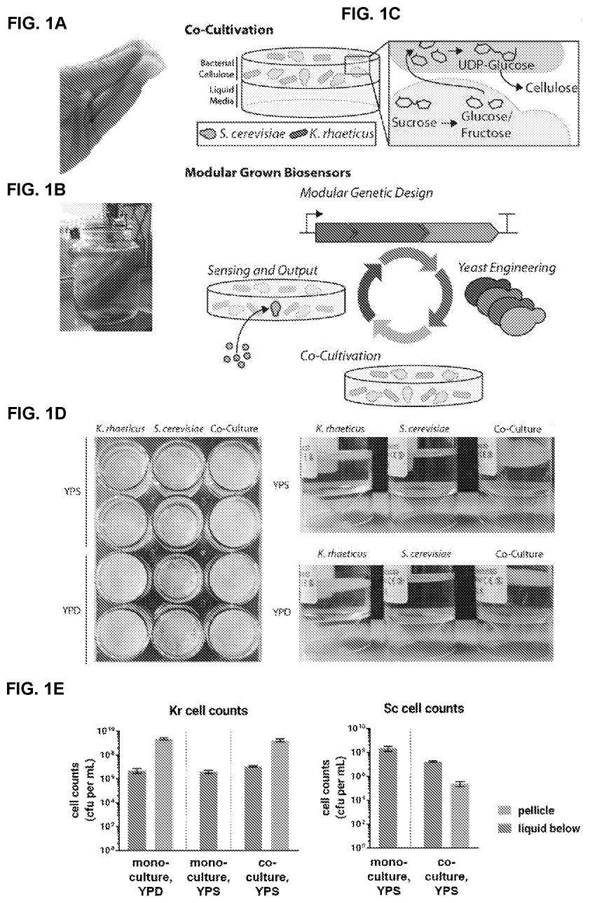

More recently, bacterial cellulose (BC) has emerged as a promising alternative model ELM system. Various species of Gram-negative acetic acid bacteria—particularly members of the Komagataeibacter and Gluconacetobacter genera—are able to produce large quantities of extracellular cellulose. When grown in static liquid culture, these BC-producing bacteria secrete cellulose, in the form of numerous individual glucan chains bundled into ribbon-like fibrils. Over the course of several days, a thick floating mat forms—referred to as a pellicle—which is composed of a network BC fibrils, within which the BC-producing bacteria become embedded (FIG. 1A).

By contrast to curli nanomaterials, BC is naturally produced at relatively high yields—reaching in excess of 10 grams per liter. Further, BC exhibits useful natural material properties, including high crystallinity, high tensile strength, biodegradability and biocompatibility. Consequently, BC has garnered interest as a feedstock material for industrial applications, including wound dressings, acoustic diaphragms for headphones and speakers, stabilizers for foams and emulsions, and scaffolds for tissue engineering and battery separators.

However, by comparison to standard synthetic biology host organisms, there is a relative paucity of genetic tools for modification of BC-producing bacteria, limiting the engineerability of BC materials. Recent years have seen an increasing number of efforts to genetically engineer BC-producing bacteria to modify BC material properties. Several early studies focused on increasing BC yields^{15,16}. More recently, BC-producing bacteria have been engineered to produce additional non-native polysaccharides, creating chitin-cellulose¹⁷ and curdlan-cellulose¹⁸ co-polymer materials. The studies herein have developed and utilized a modular genetic toolkit to engineer a newly-isolated BC-producing strain, Komagataeibacter rhaeticus¹⁹. In addition, by adding chemically modified glucose to the growth media the BC-producing bacteria is able to incorporate the subunits into cellulose and a change in BC could be achieved.

Although engineering BC-producing bacteria will likely continue to be a fruitful approach to developing BC-based ELMs, the studies presented herein were designed to test whether an alternative, co-culture approach could accelerate these efforts. Specifically, these studies set out to co-culture BC-producing bacteria with a standard synthetic biology host organism, for which a wealth of genetic tools and circuits are available. It was speculated that division of labor between BC-producing bacteria and a co-cultured host conferring novel functional properties, might expand the possibilities for BC-based ELMs. Previous work has shown that engineered E. coli can be incorporated into BC materials by manually adding cells partway through pellicle growth²⁰. This approach was used to create BC-based ELMs in which engineered E. coli could sense-and-respond to chemical inducers²¹. However, this previous approach uses manual intervention to generate co-cultures and fails to take advantage of one of the advantages of biological material production, self-assembly. By contrast, the studies presented herein provide a stable co-culture system, which enables spontaneous self-assembly and growth of BC ELMs with programmable properties.

To achieve this, the studies presented herein took inspiration from the natural source of many of the highest-yielding BC-producing bacteria, kombucha tea. Kombucha tea is a fermented beverage produced by the action of a microbial community of bacteria and yeast (FIG. 1B). Invariably this cellular consortium consists of at least one species of BC-producing bacteria and typically multiple species of yeast. There is growing interest in kombucha as a model microbial system to investigate multi-species cooperation²². One of the yeast species often found in kombucha fermentations is the model eukaryote and standard synthetic biology host organism Saccharomyces cerevisiae²³.

Therefore, the studies presented herein set out to recreate kombucha-like co-cultures of an engineerable BC-producing bacterium, K. rhaeticus, and the synthetic biology host organism S. cerevisiae (FIG. 1C). By screening various conditions, the studies presented herein established and characterized a method for stable co-culture in which K. rhaeticus and S. cerevisiae exhibited a symbiotic interaction. Using this modular co-culture system, the studies presented herein engineered S. cerevisiae strains to secrete different enzymes into BC, generating a portfolio of grown, functionalized materials. In addition, the studies presented herein demonstrate that engineered S. cerevisiae strains could be incorporated into BC, creating BC materials with tunable mechanical properties that are able to sense and respond to changes in their environment. In brief, this approach showcases the viability of co-cultures and synthetic biology tools to design, grow and test sustainable biosensors.

Establishing S. cerevisiae-K. rhaeticus Co-Culture Conditions

The first aim of the studies presented herein was to recreate kombucha-like co-cultures between S. cerevisiae and K. rhaeticus. These studies selected two engineered strains, an S. cerevisiae strain constitutively expressing green fluorescent protein (GFP) from a strong promoter (yWS195) and a K. rhaeticus strain constitutively expressing monomeric red fluorescent protein (mRFP) from a mid-strength promoter (Kr RFP). These strains were chosen to enable detection of and discrimination between strains within co-cultures through fluorescence measurements.

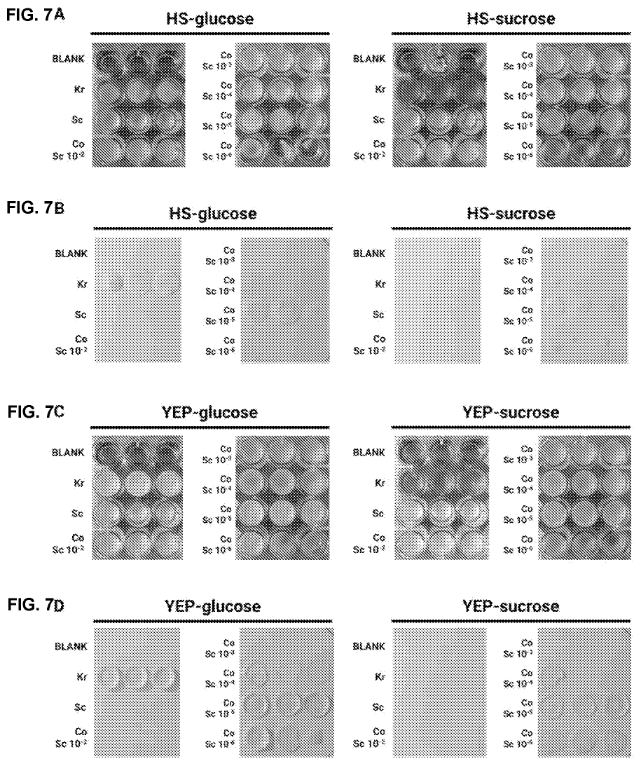

To identify conditions under which K. rhaeticus and S. cerevisiae might be efficiently co-cultured, these studies initially screened a set of co-culture conditions for growth of both strains. K. rhaeticus and S. cerevisiae were grown separately in liquid culture and then inoculated together into different media at a range of inoculation ratios. Four different culture media were selected: standard rich yeast medium with glucose (YPD) or sucrose (YPS) as the carbon source and standard medium for cultivation of BC-producing bacteria with glucose (HS-glucose) or sucrose (HS-sucrose) as the carbon source. This screen led to a number of observations regarding the growth of S. cerevisiae and K. rhaeticus (FIG. 7). Firstly, these studies found that, at low S. cerevisiae inoculation densities, co-cultures could be established in all media types. Secondly, thicker BC pellicles were obtained in yeast media (YPS and YPD) than in HS media. Thirdly, in both glucose and sucrose media, high inoculation densities of S. cerevisiae abolished pellicle formation, consistent with either nutrient competition or suppression of BC production by S. cerevisiae. Lastly, these studies found that, in mono-culture, S. cerevisiae grew well in all media types, forming a dense sediment at the base of the culture well. In contrast, in sucrose-containing media, K. rhaeticus grew poorly compared to glucose-containing media, failing to form a pellicle after 3 days. But, unexpectedly, when co-cultured with S. cerevisiae inoculated at low density, the growth of K. rhaeticus in sucrose media was substantially increased, indicating that the presence of S. cerevisiae has some stimulatory effect on the growth of K. rhaeticus.

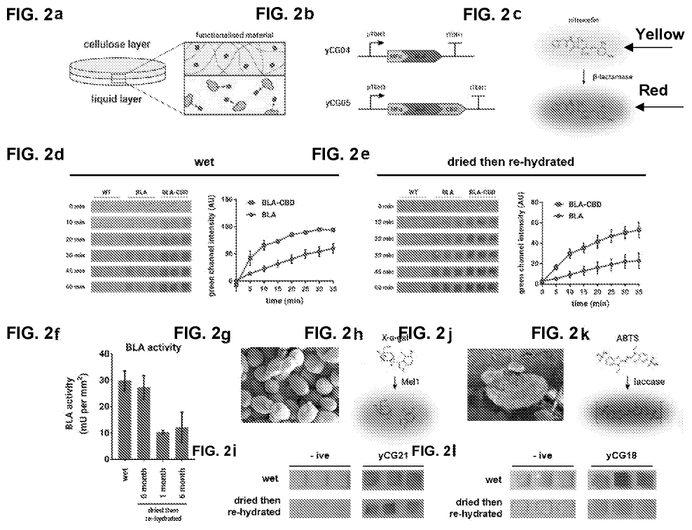

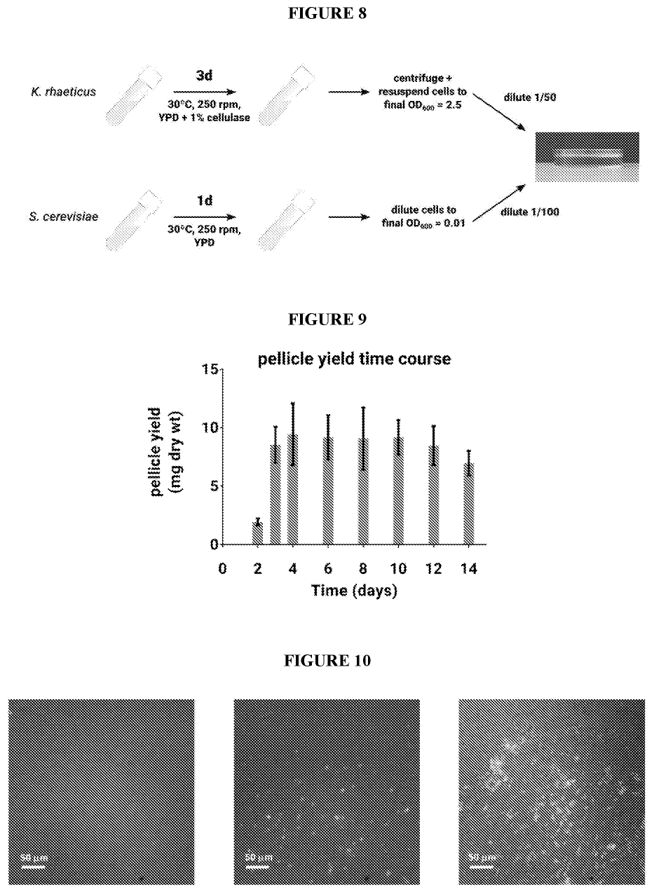

These studies next attempted to develop a standard co-culture protocol to confirm these observations which could be used for all subsequent investigations. In this approach, liquid cultures of K. rhaeticus and S. cerevisiae were grown and diluted to fixed cell densities, chosen based on this initial screen (FIG. 8). To confirm observations from this screen, these studies used this protocol to set up mono-cultures and co-cultures of each of S. cerevisiae and K. rhaeticus in YPD and YPS media (FIG. 2A). After 3 days of growth, S. cerevisiae in mono-culture had once again grown well in both media types, forming a sediment at the base of culture wells. Similarly, mono-cultures of K. rhaeticus behaved as in the screen, forming thick pellicles in YPD and no pellicles in YPS. Finally, when co-cultured, both strains were able to grow together in both YPD and YPS, as determined by the presence of thick pellicles as well as the formation of a sediment at the base of the culture well. It was again observed that the presence of S. cerevisiae in YPS medium conferred a strong growth benefit to K. rhaeticus.

Given the aim of establishing a robust method for co-culturing S. cerevisiae alongside K. rhaeticus, the observed unexpected beneficial interaction between K. rhaeticus and S. cerevisiae in sucrose media can be considered a useful trait. Specifically, since K. rhaeticus growth is dependent on the growth of S. cerevisiae, these co-culture conditions effectively ensure that K. rhaeticus cannot outcompete S. cerevisiae and thus ensure formation of a stable co-culture. Co-culture in YPS following this protocol was therefore defined as the standard co-culture condition.

This interaction likely represents either a commensal symbiotic relationship, where one partner benefits from the interaction while the other is unaffected or a parasitic symbiotic relationship, in which one partner benefits from the interaction while the other is detrimentally affected. A more desirable co-culture system might incorporate an obligate mutualistic symbiosis, where both species are unable to survive without the other. In this case, neither species can outcompete the other, resulting in a stable co-culture system. In some embodiments, such a co-dependence between a bacterial cell, such as K. rhaeticus, and a synthetic biology host cell, such as S. cerevisiae, can be engineered. For example, the synthetic biology host cell, such as S. cerevisiae, can be engineered to express one or more enzymes that metabolize a carbon source (e.g., in culture media) that otherwise is not usable by the bacterial cell, such as K. rhaeticus, to produce a carbon source that can be metabolized by the bacterial cell, such as K. rhaeticus. As another example, the synthetic biology host cell, such as S. cerevisiae, can be engineered to express one or more enzymes that degrade a molecule (e.g., in culture media) that otherwise is detrimental to the bacterial cell, such as K. rhaeticus, and thereby allow the bacterial cell to survive and grow.

Co-Culture Characterization

Having defined standard conditions for co-culture growth, these studies next attempted to characterize various properties of the co-culture system to guide BC material modification efforts. First, to determine the optimal incubation time for pellicle formation, the pellicle yield from co-cultures of S. cerevisiae and K. rhaeticus over time was measured. Pellicle formation was first detectable at low yields after 2 days, yields then plateaued at approximately 9 mg after 3 days (FIG. 9). Therefore, for all subsequent experiments, a 3-day incubation was selected for co-culture cultivation.

One challenge of the co-culture system affecting the downstream development of BC ELMs is the distribution of S. cerevisiae and K. rhaeticus between the liquid below the pellicle and the pellicle layer itself. Therefore, co-cultures were prepared and counts of viable cells obtained from the liquid and pellicle layers for both K. rhaeticus and S. cerevisiae. For comparison, viable cell counts were also obtained from mono-cultures of S. cerevisiae grown in YPS and K. rhaeticus grown in YPD and YPS. As described in greater detail in the methods section, since the degraded pellicle volume was not measured, cell counts in pellicles were estimated by assuming a fixed pellicle volume. In all conditions, the majority of K. rhaeticus cells were found in the pellicle layer, while the majority of S. cerevisiae cells were found in the liquid layer (FIGS. 2B and 2C). Although K. rhaeticus grew to similar cell densities in the liquid layer in YPD and YPS mono-cultures, the total cell density of K. rhaeticus is much lower in YPS as no pellicle was formed in this condition. K. rhaeticus reached similar estimated cell densities in both the pellicle and liquid layers when grown in mono-culture in YPD and in co-culture in YPS. By contrast, S. cerevisiae grew to a reduced cell density when co-cultured with K. rhaeticus in YPS compared to mono-culture in YPS, indicating that K. rhaeticus either competes with S. cerevisiae for some nutrient in the medium or creates conditions in the co-culture that inhibit S. cerevisiae growth. S. cerevisiae is still able to grow to reasonably high cell densities under co-culture conditions, reaching a cell density in the liquid layer of 1.78×10⁷ cells/mL (±2.42×10⁶ cells/mL).

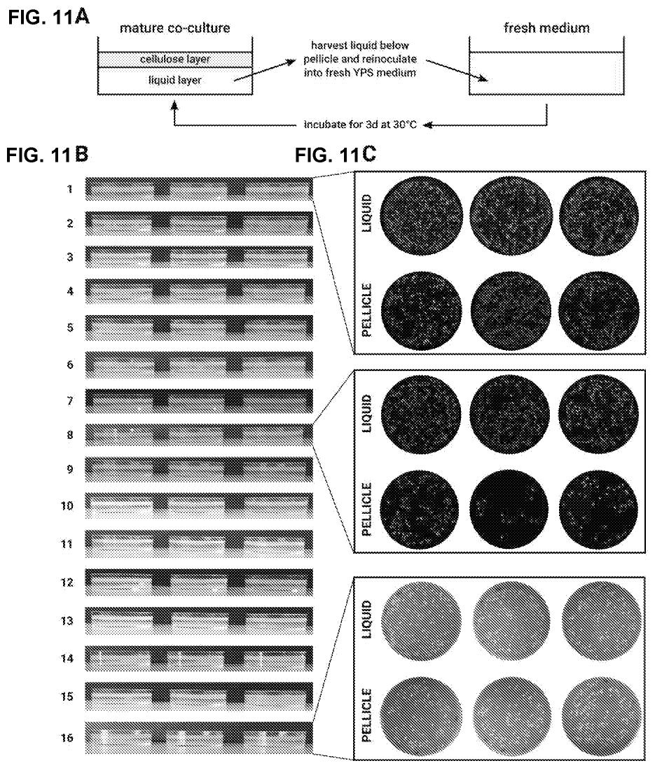

Since yeast and bacterial communities are stable over many cycles of passage during kombucha tea brewing, these studies next set out to determine to what extent the co-culture system constitutes a stable co-culture. In order to assay long-term co-culture dynamics, these studies used a serial passage approach, in which the liquid below mature pellicles was inoculated into fresh media and allowed to grow for 3 days (FIG. 11A). This process was repeated over 16 rounds (48 days). It was observed that during each round of serial passage cultures produced BC pellicles, confirming the presence of K. rhaeticus throughout serial passage (FIG. 11B). Since K. rhaeticus is unable to grow effectively in YPS medium, it was inferred that S. cerevisiae was also maintained throughout serial passage. To confirm this and to rule out the possibility of contamination with another yeast species, samples from the liquid below the pellicle and from pellicles degraded with commercial cellulase enzyme were plated onto YPD plates and the resultant colonies imaged for GFP expression (FIG. 11C). It was observed that the original S. cerevisiae strain, yWS195, was indeed maintained throughout the 16 rounds of serial passage. Additional studies have shown that, after the first round of passage, the pellicle yield is decreased and S. cerevisiae cell count increased compared to the original culture. However, for all subsequent rounds of passage, these parameters remained relatively constant.

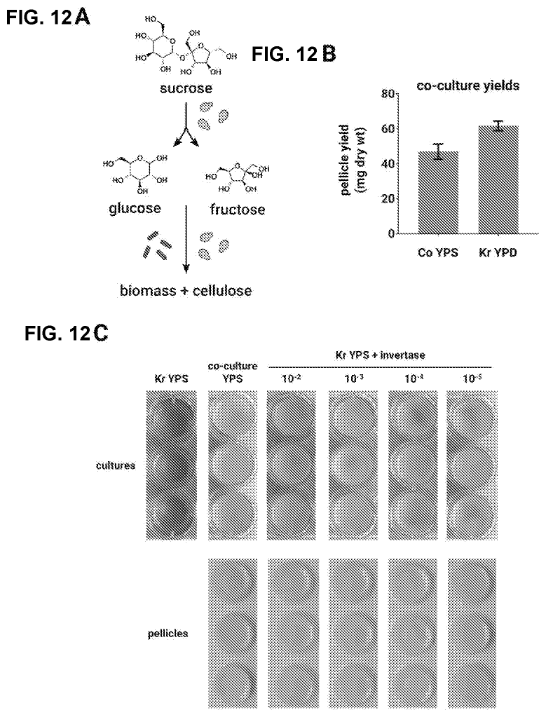

Without intending to be bound by theory, one possible explanation for the observed stimulatory effect of S. cerevisiae on the growth of K. rhaeticus in sucrose medium is that S. cerevisiae converts sucrose to a carbon source which K. rhaeticus is able to consume more efficiently. Several studies report that symbiotic interactions occur between the BC-producing bacteria and yeast in the kombucha microbial community. However, the exact nature of the interactions between kombucha microbes remains unclear. It is believed that yeasts in kombucha fermentations hydrolyze the majority of carbon source, sucrose, to form extracellular glucose and fructose through the action of the secreted enzyme invertase (FIG. 12A)²⁴. Yeasts further metabolize glucose and fructose via glycolysis, producing ethanol and creating biomass. Although BC-producing bacteria are able to grow using sucrose as a carbon source, it is believed that they mostly consume glucose, fructose and ethanol produced by the yeasts, and themselves produce acetic acid²⁵.

Without intending to be bound by theory, some studies have shown that, consistent with an interaction in which K. rhaeticus and S. cerevisiae share culture medium nutrients, pellicle yields from YPS co-cultures were reduced compared to pellicle yields from K. rhaeticus YPD mono-cultures: 47.0±4.3 mg compared to 61.7±2.7 mg, respectively (FIG. 12B).

To further explore this possibility, these studies tested whether purified S. cerevisiae invertase could enhance K. rhaeticus growth in YPS medium (FIG. 12C). As before, K. rhaeticus failed to produce a pellicle when grown in YPS. However, when grown in YPS spiked with invertase enzyme, K. rhaeticus produced thick BC pellicles after 3 days of incubation, similar to those produce under co-culture in YPS. Without intending to be bound by theory, this observation suggests that the secretion of invertase by S. cerevisiae, resulting in the accumulation of extracellular glucose and fructose, may underlie the observed symbiotic effect in these co-culture conditions.

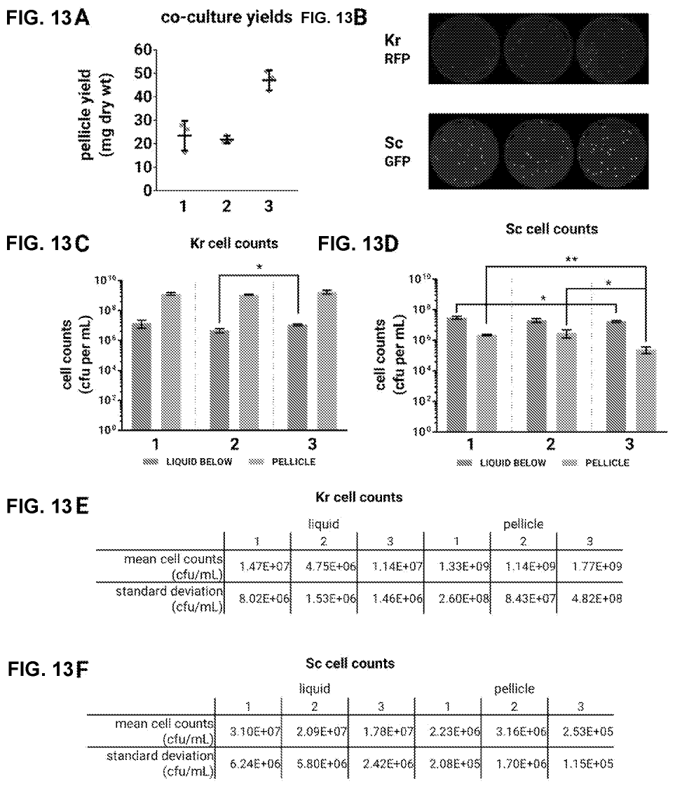

Finally, to give an idea of the robustness of the co-culture methods provided herein, these studies set out to determine the reproducibility of co-culture properties. To achieve this, identical co-cultures were prepared following the standard protocol on three separate occasions and two parameters were measured: pellicle yields and cell counts. These two parameters were chosen as they are likely to significantly impact any downstream applications. For instance, if S. cerevisiae is engineered to secrete proteins into the pellicle, both the cell density of S. cerevisiae in the co-culture and the pellicle yield will strongly influence the final titers of secreted protein. It was found that pellicle yields tended to be consistent within triplicate samples, but variable between co-cultures set up on different occasions (FIG. 13A). As before, cell counts were determined by plating liquid from below the pellicle and degraded pellicle samples onto selective media and imaging plates under fluorescence (FIG. 13B). Estimated cell counts for K. rhaeticus were consistent in the pellicle layer, where the majority of cells were detected, but varied by up to an order of magnitude in the liquid layer (FIGS. 13C and 13E). Similarly, S. cerevisiae cell counts were consistent in the liquid layer, where the majority of cells were detected, but more variable in the pellicle layer (FIGS. 13D and 13F).

The propensity for S. cerevisiae to grow in the liquid layer rather than the pellicle layer might preclude the utility of co-cultured S. cerevisiae for certain applications—for example, controlling pattern formation or creating biosensor materials. In addition, fluorescence microscopy showed that S. cerevisiae cells that were present in the pellicle layer exhibited a highly-variable distribution across the pellicle (FIG. 10). The co-culture systems provided herein take into account that applications such as autonomous material patterning require an even distribution of engineered cells across the material.

Engineering BC Material Functionalization

Having developed conditions for growth of co-cultures, these studies next attempted to take advantage of the wealth of biotechnological tools developed for S. cerevisiae to confer new biological functions to BC materials. Since S. cerevisiae is well-known as an effective recombinant protein secretion host, these studies first asked if S. cerevisiae strains could be engineered to secrete proteins and thereby functionalize BC materials (FIG. 2A). To test this, these studies selected a protein that had been previously-demonstrated to be secreted from S. cerevisiae, the β-lactam hydrolyzing enzyme, β-lactamase (BLA). Specifically, the catalytic region of E. coli TEM1 BLA, lacking the native signal peptide was chosen. Using the yeast toolkit (YTK) system²⁷, the BLA catalytic region was cloned downstream of the S. cerevisiae mating factor alpha (MFα) prepro secretion signal peptide under the control of a strong constitutive promoter (pTDH3) to create strain yCGO4 (FIG. 2B). However, since the BC layer constitutes only a fraction of the total volume of the co-culture, only a fraction of the secreted BLA would be expected to be incorporated into the BC layer. Therefore, a second strain (yCG05) was engineered in which a cellulose-binding domain (CBD) was fused to the C-terminus of BLA (FIG. 2B). It was hypothesized that addition of a CBD might increase the proportion of secreted protein bound within the BC layer and so improve the efficiency of functionalization. While numerous CBDs have been described in the literature, CBDcex²⁸ (the 112 amino acid region from the C-terminus of the Cex exoglucanase from Cellulomonas fimi) was chosen based on previous work demonstrating its ability to bind BC¹⁹.

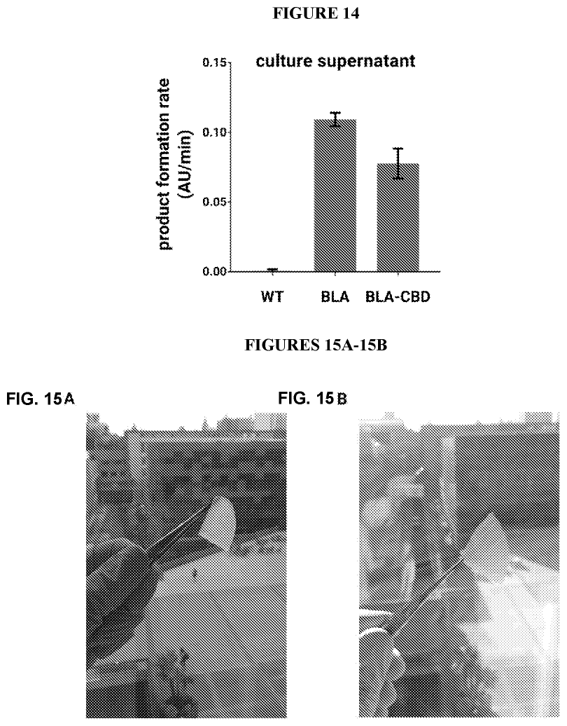

To confirm that the engineered strains were able to secrete functional BLA enzyme, wild type, yCGO4 and yCGO5 S. cerevisiae strains were grown in mono-culture in YPS medium. Supernatants were harvested from these cultures and then screened for BLA activity using the colorimetric nitrocefin assay (FIG. 2C). Supernatants from both yCGO4 and yCGO5 strains showed increased β-lactamase activity compared to that of the WT strain (FIG. 14). The activity detected from the BLA-CBD secreting strain, yCG05, was reduced compared to the BLA secreting strain, yCG04. Since this assay was performed using undiluted supernatants from 24-hour cultures, the enzyme activity will be affected by multiple factors. Therefore, the observed decrease in β-lactamase activity for the BLA-CBD secreting strain could be due to decreased growth rate, decreased secreted protein yields or an effect of fusion of the CBD to BLA enzyme decreasing its activity by causing steric hindrance, for example.

Having demonstrated that the engineered S. cerevisiae strains can secrete active BLA, these studies next attempted to test whether they could be co-cultured with K. rhaeticus to produce a grown, enzyme-functionalized material. Co-cultures were prepared with wild type, BLA-secreting (yCG04) or BLA-CBD-secreting (yCG05) strains and the resultant unprocessed, wet pellicles were screened for β-lactamase activity. While pellicles from co-cultures with WT S. cerevisiae showed no BLA activity, a clear signal was observed with pellicles from co-cultures with BLA-secreting and BLA-CBD-secreting strains (FIG. 2D), demonstrating that engineered S. cerevisiae can indeed direct BC functionalization. Fusion of the CBD to the BLA C-terminus resulted in an increase in the observed β-lactamase signal. Therefore, although fusion of the CBD resulted in a decreased yield of secreted BLA in mono-culture, under co-culture it results in an increase in the proportion of secreted enzyme that becomes incorporated into the pellicle layer and so a greater degree of BC functionalization.

Since native BC pellicles have a water content of ˜99%, these studies attempted to determine if enzyme-functionalized materials could be dried and re-hydrated without eliminating BLA activity. To test this, pellicles produced by co-culturing K. rhaeticus with WT, BLA-secreting (yCG04) and BLA-CBD-secreting (yCG05) S. cerevisiae strains were dried by sandwiching them between sheets of absorbent paper towel to create thin, paper-like materials (FIG. 15). Dried pellicles were then rehydrated and screened for β-lactamase activity. Dried pellicles from co-culture with BLA-secreting yCGO4 and BLA-CBD-secreting yCGO5 strains demonstrated clear BLA activity (FIG. 2E). Again, a greater signal was observed in pellicles from co-culture with the BLA-CBD secreting strain compared to the BLA secreting strain.

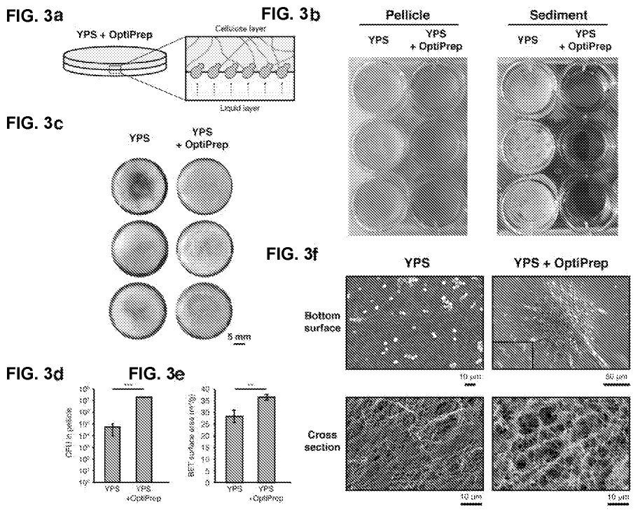

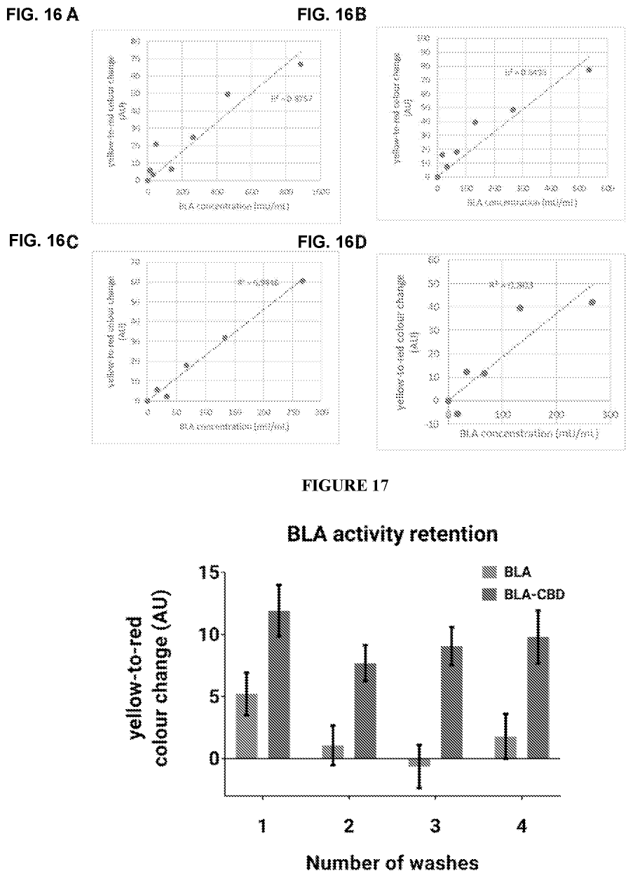

To enable comparison of the absolute levels of β-lactamase activity between wet pellicles and dried pellicles, the nitrocefin assay was repeated alongside standard curves with a commercial BLA enzyme (FIG. 16). The drying process had little effect on the activity of BLA in the material: 29.8±3.7 mU/mm² before drying and 27.3±4.4 mU/mm² after (FIG. 2F). Further, the assay was performed following storage of functionalized BC for 1 month and 6 months at room temperature without desiccant. Remarkably, after long-term storage under ambient conditions, the pellicles retained β-lactamase activity, although activity was reduced to around a third of the original level (FIG. 3F). This shows that functionalized materials can be grown and stored at room temperature, retaining their activity for later rehydration and deployment.

As the BLA enzyme is passively incorporated within the BC matrix by diffusion and the BLA-CBD fusion is specifically bound through the CBD-cellulose interaction, it might, be anticipated that BLA enzyme could leach out of the BC material over time, while BLA-CBD would remain bound stably. To test this, dried pellicles functionalized with BLA and BLA-CBD were subjected to multiple rounds of washes in PBS buffer and then assayed for β-lactamase activity (FIG. 17). The activity of β-lactamase in BLA-functionalized pellicles fell sharply after washing, by contrast, BLA-CBD-functionalized pellicles retained a greater proportion of their original β-lactamase activity after washing. This observation is consistent with the CBD providing a specific, stable binding interaction between the enzyme and cellulose. The choice of whether to include a CBD or not therefore offers flexibility for applications which may require stably functionalized materials or materials able to leach a functional species.

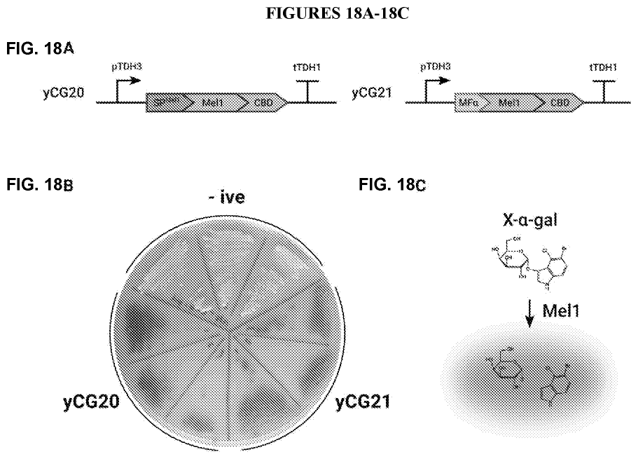

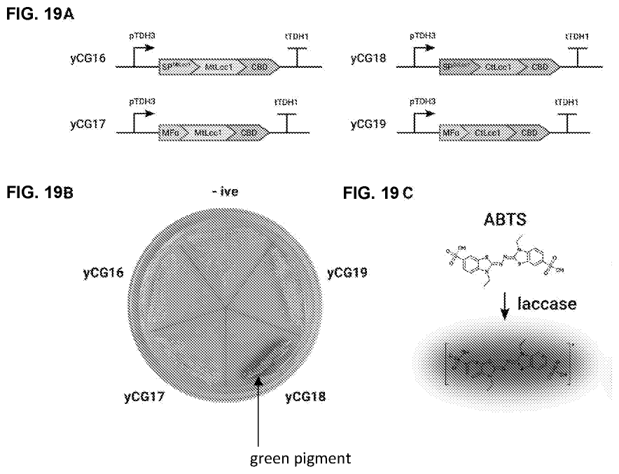

Having demonstrated that the co-culture approach enables BC functionalization with β-lactamase, these studies set out to determine whether BC can be functionalized with other enzymes. A native S. cerevisiae secreted protein, the alpha-galactosidase enzyme Mel1 for which a simple colorimetric activity assay is available, was selected for these studies. In addition, two laccase enzymes, previously engineered to be expressed and secreted from S. cerevisiae²⁹, were selected. Laccases catalyze one-electron oxidations, typically possessing a broad substrate range and have attracted interest for numerous industrial applications, such as in textile and food processing, in the pharmaceutical and chemical industries, in biofuel cells, and in the degradation of environmental pollutants³⁰. The YTK cloning system was used to clone S. cerevisiae Mel1 and fungal laccases from Myceliophthora thermophila (MtLcc1) and from Coriolopsis troggi (CtLcc1). Variants of each protein were generated with either the native secretion signal peptide or the S. cerevisiae MFα signal peptide, all of which also possessed a C-terminal CBD (FIGS. 18A and 19A). Strains secreting Mel1 fused to the MFα signal peptide and CtLcc1 fused to its native signal peptide exhibited the highest secretion yields in colorimetric plate-based assays (FIGS. 18B and 19B). These strains were then co-cultured with K. rhaeticus and pellicles assayed for enzyme activity by colorimetric assays. Laccase and α-galactosidase activities were detected in native, wet pellicles (FIGS. 3I and 2L), demonstrating that the co-culture method enabled BC functionalization with active CtLcc1 and Melt. Further, pellicles functionalized with either CtLcc1 or Mel1 retained activity after drying and re-hydration, showing that dried functionalized BC materials can be generated with enzymes other than β-lactamase.

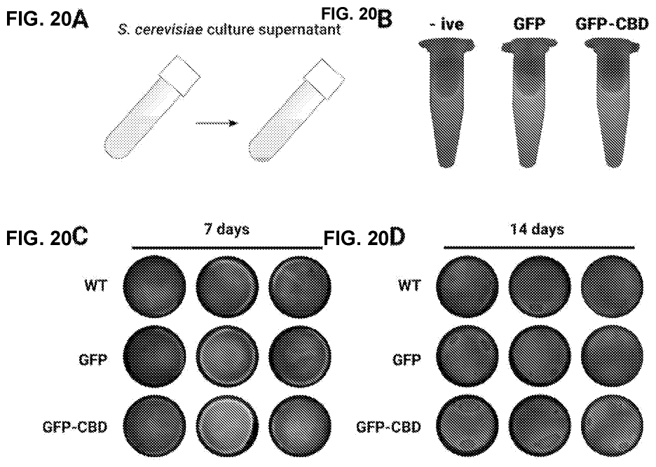

Finally, these studies explored whether BC could be functionalized with a more challenging target protein which is not secreted from its native host, green fluorescent protein (GFP). Previous attempts to secrete GFP from S. cerevisiae have yielded mixed results, with some studies reporting inefficient secretion and others reporting low milligram per liter secretion yields^{31,32}. Here, these studies again employed the YTK system to generate strains secreting GFP and GFP fused to CBDcex. Both GFP and GFP-CBD were fused to the S. cerevisiae MFα secretion signal peptide and expressed from a strong constitutive promoter. It was found that both GFP and GFP-CBD could be detected in the culture supernatant after 48 hours growth in shake-flask, indicating successful secretion (FIGS. 20A and 20B). However, co-culture of these strains with K. rhaeticus produced no detectable increasing in BC pellicle fluorescence, even after incubation for extended periods of time (FIGS. 20C and 20D). Therefore, although low levels of GFP secretion were detectable from shake-flask cultures, the relative lower cell density of S. cerevisiae under co-culture conditions likely accounts for the low apparent yield of secreted protein.

In some embodiments, the co-culture approach for BC functionalization using engineered S. cerevisiae strains to functionalize BC may be limited to cases where proteins of interest can be efficiently secreted or are highly-active at low yields. In addition, in some embodiments, the applicability of enzyme-functionalized BC materials produced through this method may be limited to conditions under which functionalizing proteins and CBDs remain active. Finally, since sterilizing BC materials without inactivating functionalizing proteins poses a challenge, in some embodiments, potential applications may use BC materials containing live engineered cells.

The primary motivation of these efforts was to demonstrate that a co-culture approach enables self-assembly of BC materials with engineered functional properties. However, methods to attach enzymes to materials—also known as ‘enzyme immobilization’—have potential applications in a number of industries. Broadly, enzyme immobilization is used to improve the cost-effectiveness of industrial biocatalysts, facilitating purification of the product from the enzyme and often improving the stability and reusability of the enzyme. Immobilized enzymes are used in a variety of industrial processes: lactase in lactose-free milk production³³, glucose isomerase in high-fructose corn syrup production³⁴ and lipases in the interesterification of food fats and oils³⁵. Moreover, BC has been proposed as a suitable material substrate for enzyme immobilization³⁶⁻³⁸. The approach provided herein enables sustainable, self-assembled enzyme immobilization under mild conditions of BC-materials which may have potential utility in decontamination of soil and wastewater from β-lactam antibiotics^{39,40}. Therefore, 0-lactamase-functionalized BC materials could be applied to the bioremediation of these environments. Similarly, given their numerous potential uses, BC materials functionalized with laccase enzymes could be applied in areas such as bioremediation, pharmaceutical and chemical industries or textile and food processing. These and other applications might be facilitated by the fact that enzyme-functionalized BC materials can retain activity following drying and re-hydration after long-term storage.

Incorporating S. cerevisiae Cells within BC Materials

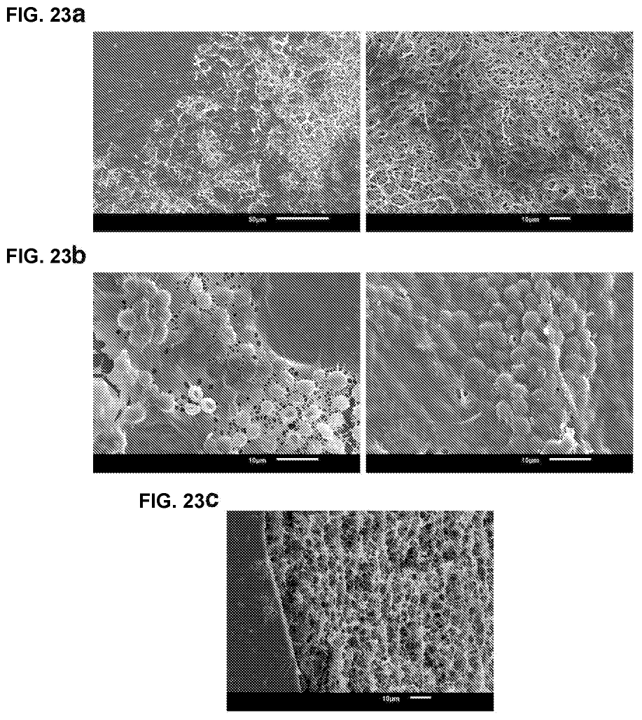

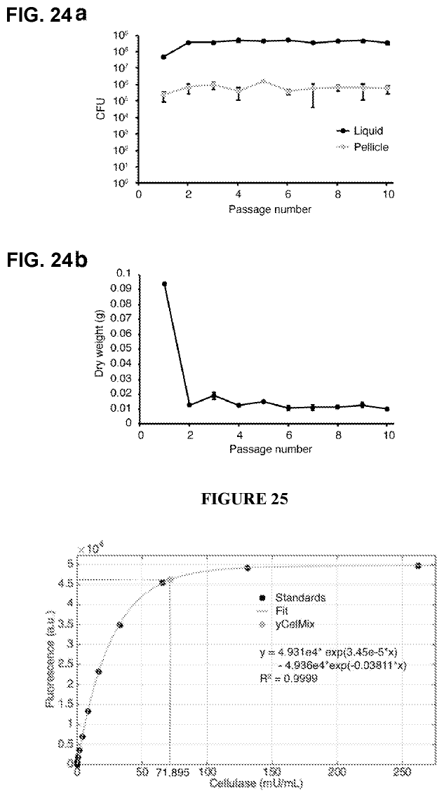

The majority of S. cerevisiae cells in the co-cultures are found in the liquid layer below the pellicle, which has to be taken into account when generating biological ELM engineering efforts. Consequently, the studies provided herein attempted to develop a co-culture method that would enable efficient incorporation of S. cerevisiae cells within BC. S. cerevisiae settles to the bottom of culture vessels because the density of the cells is greater than that of water: ˜1.11 g/mL compared to 1 g/mL²⁶. Therefore, it was hypothesized that, if the density of the culture medium were increased to >1.11 g/mL, S. cerevisiae cells would float to the surface rather than sinking and so become incorporated into the newly-forming pellicle at the air-water interface (FIG. 3a). Inspired by the work of Bryan et al. using Percoll as a density modifying agent to adjust the density of growth medium, OptiPrep, a metabolically inert aqueous solution of 60% iodixanol with a density of 1.32 g/mL, was used to increase the density of YPS well above 1.11 g/mL. Since OptiPrep has low viscosity and osmolarity, survival and growth of K. rhaeticus and S. cerevisiae should remain similar to incubation in YPS. In order to confirm these hypotheses, co-cultures were prepared in standard YPS medium and in YPS supplemented with 45% OptiPrep (v/v) and images taken of the resultant cultures. Under both conditions BC pellicles formed with little apparent difference in their thickness and yield (FIG. 3b). When pellicles were removed, there was a complete absence of sediment in the YPS-OptiPrep medium, in contrast to the dense sediment observed in standard YPS medium (FIG. 3b). Further, compared to the smooth, homogenous surface of pellicles formed in YPS, pellicles formed in YPS-OptiPrep had a speckled appearance (FIG. 3c). Taken together, these results suggest that addition of OptiPrep to the co-culture medium increases the density such that S. cerevisiae cells become buoyant and are therefore incorporated into the pellicle layer. To confirm this observation, estimated counts of S. cerevisiae cells within pellicles from co-cultures with or without OptiPrep were determined (FIG. 3d). Indeed, addition of OptiPrep resulted in a ˜339-fold increase in estimated cell count from 5.50×10⁴ cfu/mL (±4.58×10⁴) to 1.87×10⁷ cfu/mL (±1.15×10⁶). Addition of OptiPrep to co-culture medium therefore represents a simple approach to direct the incorporation of S. cerevisiae cells into the growing BC material layer under static conditions. Moreover, it was discovered that pellicle formed in the presence of OptiPrep exhibits a larger total surface area, as determined by Brunauer-Emmett-Teller (BET) measurement. Such increase would benefit enzyme-based functionalization as the internal catalytic surface in cellulose network is expanded. These observations were confirmed in the scanning electron microscopy (SEM) images taken on dried pellicles (FIG. 3f and FIG. 23). Yeast cells in pellicle grown in YPS were loosely attached to the bottom surface and were easily washed away, while they are incorporated into foci covered by cellulose fibrils in the presence of OptiPrep. In addition, enlarged macroporous structures were observed in pellicle cross section, possibly because of the fermentation gas generation happening in proximity with cellulose matrix formation. A disadvantage of this approach is the prohibitive cost of OptiPrep, which is likely to be a significant barrier to cost-effective scale-up. In some embodiments, alternative approaches—for instance, using a cheaper density-modifying reagent or genetically-encoding S. cerevisiae incorporation into the pellicle—are used.

Modifying BC Material's Physical Properties with Secreted Enzymes

In nature, living cells can modify or remodel their surrounding non-cellular matrix using various secreted enzymes. Such process plays an important part in the development and differentiation of multicellular organisms as well as disease progression. Inspired by the role of proteases in orchestrating the dynamics of protein-based extracellular matrix, these studies explored whether one could modify the cellulosic matrix in the pellicles with cellulases secreted from the yeast cells, thus change the physical properties of the BC living materials.

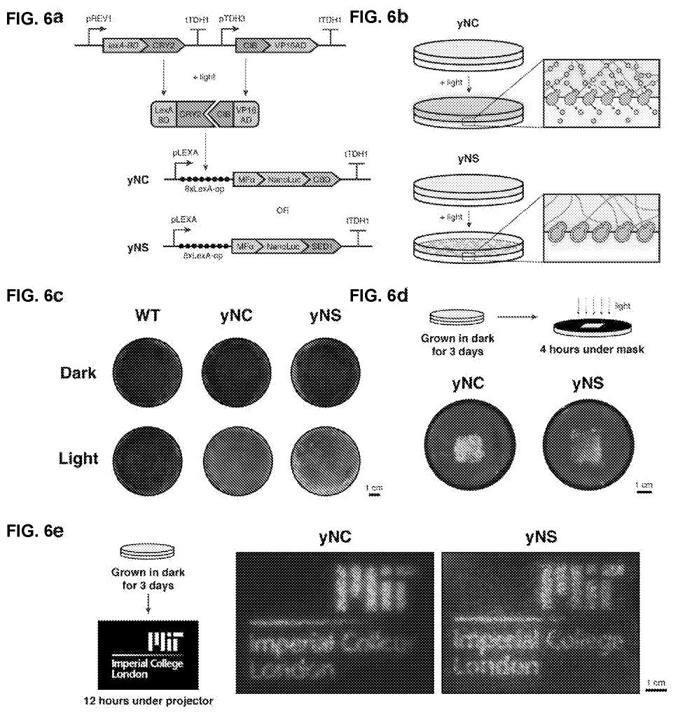

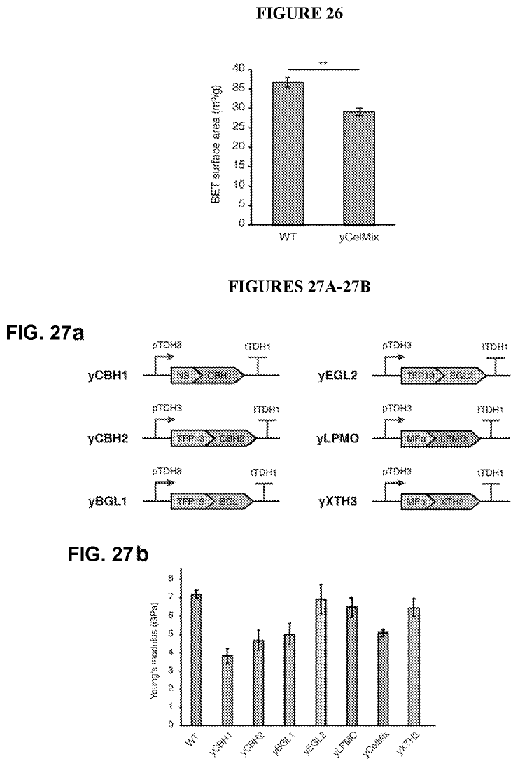

Metabolic engineering of S. cerevisiae for cellulose degradation through expressing fungal cellulases has become a well-established field in past decades. In this study, a yeast strain, yCelMix, in which cellobiohydrolases (CBH1 and CBH2), endoglucanase (EGL2), β-glucosidase (BGL1), and lytic polysaccharide monooxygenases (LPMO) are optimized to be secreted simultaneously for synergistic cellulose degradation, was constructed. (FIGS. 6a and b, FIG. 9). After 2 days static incubation, pellicles with cellulase secreting yeast showed slightly decreased dried weight (FIG. 6c), indicating that the rates of S. cerevisiae enzyme secretion and action are slower than the rate of K. rhaeticus cellulose biosynthesis. Moreover, extending the incubation time to five days did not further reduce the cellulose mass, potentially due to the limited diffusion of enzymes, low pH, local accumulation of reaction products, and cellulase deactivation at the air-liquid interface in static culture. The total surface area of the yCelMix pellicle is smaller than that of the WT pellicle, with mechanism that remains to be elucidated (FIG. 26).

Despite the cellulose yield is not deeply impacted by cellulase secretion, the mechanical properties of the pellicles are significantly altered. The stress-strain curves from tensile test demonstrates a clear difference between WT pellicle and yCelMix pellicle (FIG. 6d). Specifically, while both being brittle (tensile strength equals fracture strength), yCelMix pellicle can only sustain 45.7 MPa while WT pellicle can bear 98.3 MPa of stress before fracturing (FIG. 6e). Also, yCelMix pellicle can only be stretched to about half of the amount WT pellicle can elongate at break (FIG. 6f). And it is clear that secreted cellulases successful reduce the stiffness of the cellulose matrix, lowering the Young's modulus from 7.2 GPa (WT) to 5.1 GPa (yCelMix) (FIG. 6g and FIG. 27). Although the studies provided herein have not investigated the detailed mechanisms of enzyme modifying cellulose matrix in co-culture, these results provided by the studies presented herein support that continuity of the cellulose fibrils and integrity of network structure are essential for the strength and stiffness of bacterial cellulose materials.

The weakening of microstructure in yCelMix pellicle is also reflected by its rheological properties. In the strain sweep experiment (FIG. 6h), both pellicles show gel-like behavior at low strain as G′ always dominates over G″. The G′ and G″ of yCelMix pellicle are both lower than those of WT pellicles', again demonstrating that cellulases reduce the stiffness of the BC material (FIG. 6h) in a frequency-independent fashion (FIG. 6i). As the applied strain increases beyond the G′ G″ crossover point, where micro cracks accumulate and major rupture appear, the pellicles switch to a viscoelastic liquid and start to flow. It is worth-noting that yCelMix pellicle has an earlier onset of crossover, indicating a faster breakdown of network structure in the matrix. In addition, the more pronounced G″ maximum in yCelMix pellicle (FIG. 6i) demonstrates that deformation energy is converted into friction heat from the free broken fibrils near the micro cracks, which originates from a disintegrated and weaker cellulose network. These results suggest that secreting cellulases can effectively weaken the mechanical and viscoelastic properties of BC materials, complementing the recent studies showing CBD additives can enhance the strength of cellulosic materials.

Engineering Sense-and-Response BC Materials

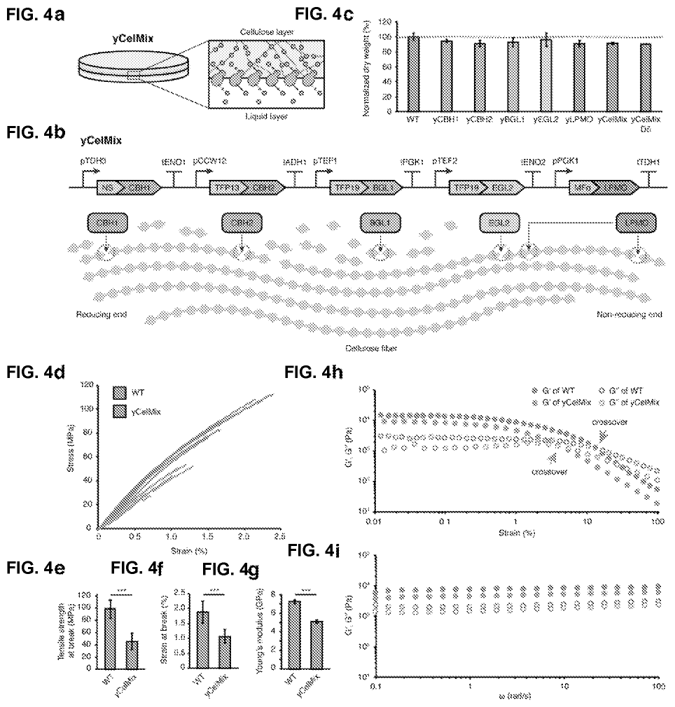

Another of the advantageous properties of natural biological materials is their ability to sense-and-respond to changes in their external environment. A variety of genetic circuits have been engineered in S. cerevisiae enabling biological, chemical, and physical stimuli to drive changes in gene expression. Therefore, these studies attempted to determine whether such engineered biosensor S. cerevisiae strains could be incorporated into BC to create materials able to sense-and-respond to environmental stimuli (FIG. 4A). To begin with, these studies selected a chemically-inducible system from the literature, in which addition of the estrogen steroid hormone β-estradiol leads to activation of transcription from a target promoter (FIG. 4B). These studies generated an S. cerevisiae strain (GPY093) in which Z3EV is expressed from a constitutive weak strength promoter and GFP is under the control of a β-estradiol (BED) responsive promoter—a Gall promoter containing six repeats of the Zif268 target sequence (FIG. 4B).

In order to generate BC materials in which the GPY093 biosensor strain was incorporated within the BC matrix, co-cultures of K. rhaeticus and wild-type or GPY093 S. cerevisiae were prepared in YPS-OptiPrep medium. The resultant pellicles were rinsed and then incubated in fresh medium in the presence or absence of BED for 24 hours. By contrast to pellicles grown with wild-type S. cerevisiae, addition of BED to pellicles containing GPY093 S. cerevisiae yielded a strong GFP signal (FIG. 4C), demonstrating that this co-culture approach enables self-assembly of BC materials able sense-and-respond to environmental stimuli.

A variety of other S. cerevisiae biosensor strains have been engineered to screen and detect a range of environmental pollutants and pathogens⁴¹⁻⁴³. This approach could, therefore, be used to create grown biosensor materials for on-site screening of medical or environmental samples. There is increasing concern over environmental pollution with endocrine disruptors/estrogen hormones, including, by way of non-limiting example, BED. In some embodiment, biosensor BC materials are useful in the detection of BED in the environment.

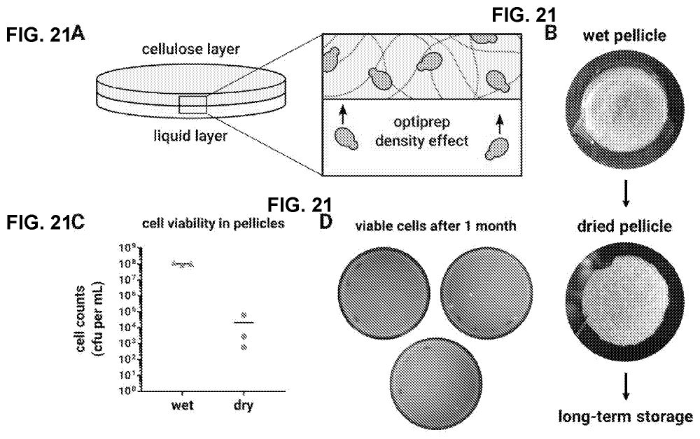

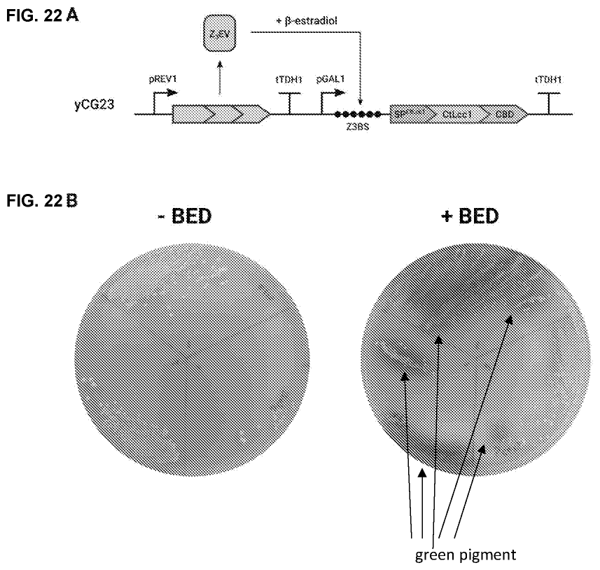

However, for this approach to be feasible, biosensor materials would have to be stored without losing their functionality. Therefore, these studies attempted to determine if S. cerevisiae cells incorporated into BC materials could remain viable after drying and long-term storage (FIG. 4D). To test this, co-cultures were prepared in YPS-OptiPrep medium to create pellicles into which the GFP-expressing S. cerevisiae Sc GFP strain was incorporated (FIG. 21A). The resultant pellicles were then dried and stored at room temperature under ambient conditions (FIG. 21B). Wet and dried pellicles were degraded enzymatically, plated onto selective medium and cell counts of S. cerevisiae determined. Although drying resulted in a sharp decrease in the viability of cells within BC materials, a significant number of viable cells remained (FIG. 21C). In addition, although at low levels, viable S. cerevisiae cells could be detected in pellicles after 1 month of storage at room temperature under ambient conditions (FIG. 21D). While the majority of S. cerevisiae cells within pellicles did not remain viable after drying, even small numbers of viable cells may be sufficient for biosensor materials to remain functional. Since biosensor cells can be incubated in fresh culture medium (FIG. 4E), an initially small population of viable cells is therefore able to grow and generate a large response. Biosensor pellicles containing GPY093 were grown, dried and incubated in fresh medium for 24 hours in the presence of absence of BED. Pellicles containing GPY093 S. cerevisiae yielded a clear GFP signal in the presence of BED (FIG. 4F). Further, re-hydrated dried pellicles stored under ambient conditions for 4 months yielded a detectable GFP signal in the presence of BED (FIG. 4G). While these sense-and-response functions use the addition of fresh medium, it may be possible to screen diverse sample types by supplementing with concentrated nutrient stocks. This approach has been employed previously, enabling S. cerevisiae biosensor strains to function in blood, urine and soil⁴¹.

As mentioned above, S. cerevisiae strains have been engineered to sense-and-respond to numerous other biological, chemical, and physical stimuli. One class of S. cerevisiae biosensors employs the G protein coupled receptor (GPCR) family of receptors. GPCRs are membrane protein receptors that share a common basic structure but are able to detect a remarkable range of different chemical and physical stimuli. S. cerevisiae possesses a native GPCR signaling cascade, which it uses to sense-and-respond to mating pheromones. By transplanting heterologous GPCRs into this pathway, biosensors with novel targets can be generated. To demonstrate that this approach is compatible with GPCR-based signaling, co-cultures were prepared with a S. cerevisiae biosensor strain. This biosensor strain detects the S. cerevisiae mating factor alpha (MFα) peptide through the native Ste2 GPCR, activating GFP expression in response. Pellicles into which yWS890 and wild type S. cerevisiae had been incorporated were grown, dried and re-hydrated in the presence of absence of MFα. Biosensor pellicles exhibited a clear increase in GFP signal in the presence of MFα, indicating that the GPCR-based biosensor strain does indeed function well using this grown biosensor approach (FIG. 4H). Although in some cases it remains a significant challenge to successfully transplant heterologous GPCRs into the native S. cerevisiae cellular machinery, numerous studies have succeeded in developing novel GPCR-based S. cerevisiae biosensors^{41,43}. Using this approach, these and additional embodiments of the biosensor strains could, therefore, be employed to create a panel of grown biosensor materials.

Biosensor strains serve to sense external stimuli and provide a convenient readout, in this case in the form of fluorescent protein expression. However, living materials are also able to dynamically remodel and adapt their functional properties in response to changes in their environment. These studies took advantage of the modularity of the YTK to engineer an S. cerevisiae strain secreting the laccase CtLcc1 under control of the BED-inducible promoter, yCG23 (FIG. 22). Laccases have been previously demonstrated to enable the degradation of BED for the purpose of wastewater bioremediation. This co-culture approach enabled self-assembly of BC materials loaded with yCG23 which could in simultaneously detect the presence of BED and in response secrete active laccase enzyme (FIG. 4J). A similar system was previously engineered with E. coli curli biofilm materials, enabling detection of the presence of heavy metal ions and directing their sequestration in response. Just as proposed in that report, this approach could be used to create living materials loaded with ‘sentinel’ cells, engineered to screen for a panel of environmental pollutants and toxins and to sequester or degrade them in response.

Spatial Patterning of Catalytic Living Materials

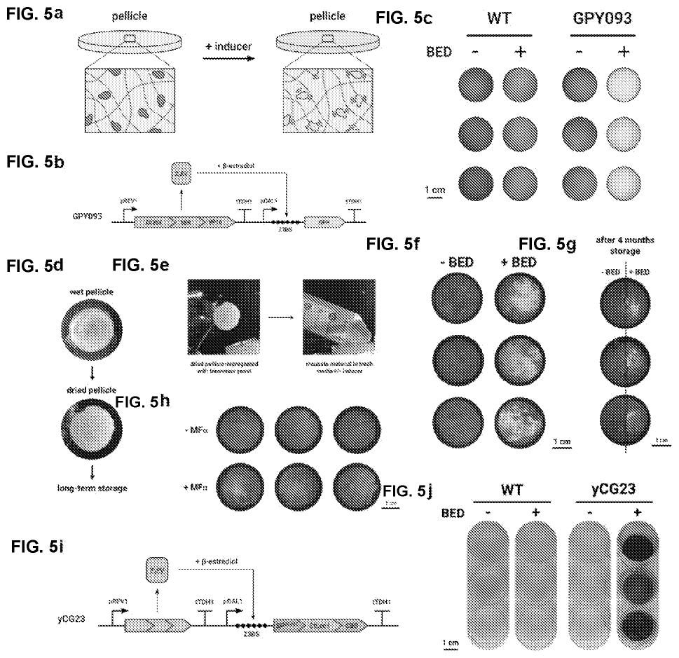

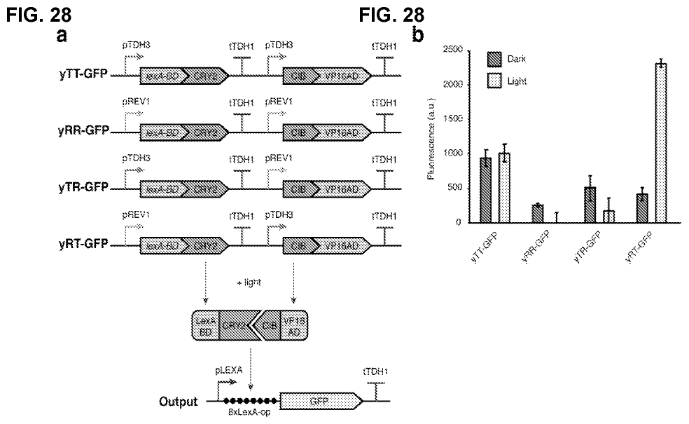

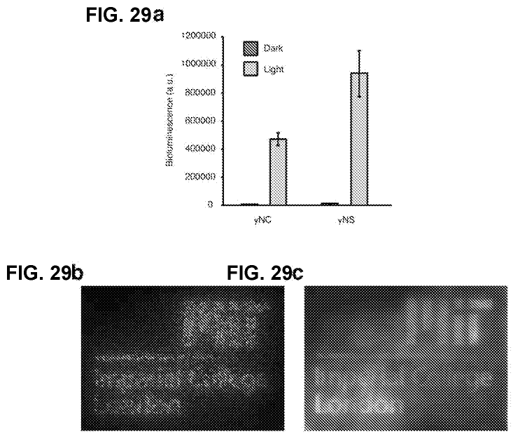

Building upon the fact that living yeast cells can sense and respond to chemical inputs in the co-culture system, these studies further investigated the possibility of using other modalities of inputs to program the living materials. Optogenetic tools in bacteria such as E. coli have enabled high-resolution spatial patterning of microbial biofilm using light-inducible control of protein-protein interactions. Inspired by the recent advances in optical dimerizers in yeast, these studies started the implementation by using a blue light sensing system that is based on the CRY2/CIB transcription system. To achieve high activation and low background expression, these studies first investigated the importance of promoter strengths in driving the expression of the DNA binding component (LexA-CRY2) and the activation component (VP16-CIB1) (FIG. 28). It was found that the best light-induced GFP expression profile is achieved by driving LexA-CRY2 driven with a weak constitutive promoter (pREV1) and driving VP16-CIB1 by a strong constitutive promoter (pTDH3) (FIG. 28b). Following this finding, these studies constructed two yeast strains by substituting the GFP output with an enzyme, NanoLuc, with C-terminal fusions partners SED1 (yNS strain for NanoLuc surface display) and CBD (yNC strain for NanoLuc secretion and binding to cellulose) (FIGS. 5a and b). The luciferase output outperforms the previous fluorescence output as the catalytic step greatly increases the sensitivity and reduces background noise (FIG. 29a). To test if BC materials that respond to an optical input could be built, these studies grew yNC and yNS co-cultures in YPS+OptiPrep with or without exposure to white light. After 3 days incubation in light, both yNC and yNS pellicles showed high bioluminescence when substrate was applied, while their control pellicles in dark showed nearly zero luciferase activity (FIG. 5c). Specifically, the yNC pellicle exhibits even-distributed NanoLuc activity across the entire pellicle surface whereas the yNS pellicle shows localized foci corresponding to yeast cell distribution. This demonstrates how one can fine-tune the local distribution of enzymatic functionalization in BC materials through switching between cell surface display and secretion modes.

These studies further explored the responsiveness of yNC and yNS pellicles to light patterning created by masking and projecting. These “living films” were grown in dark for 3 days before exposure. For mask patterning, the pellicles were covered by a black foil piece with a square carved out in the center, where bioluminescence soon appeared after 4 hours of development under a LED lamp (FIG. 5d). Both yNC and yNS pellicles showed bright foci within the pattern, likely because there was insufficient time for the NanoLuc diffusing through the cellulose matrix away from the yNC cells. For projector patterning, these studies put the living films in an incubator in which they were exposed to a complicated pattern projected from an LED projector mounted on the top. In both yNC and yNS pellicles, the projected patterns were faithfully mirrored to the final luciferase activity output (FIG. 5e), as yNC produced a more diffused and blurrier pattern in comparison with yNS. The areas that were more densely patterned showed limited resolution, possibly due to the internal scattering originated from the opaque cellulose matrix. In addition, changing the yeast cell density by either slowing down their growth rate or increasing incubation time in dark can either decrease (FIG. 29b), or increase (FIG. 29c) the resolution of the projected patterns, respectively. These knobs, along with different patterning modes (light mask for rapid, large scale prototyping and projection for complicated patterning), provide a basic toolset for optogenetic functionalization of BC materials. In some embodiments, this system is readily expandable through incorporating other orthogonal light dimerizer system linked to various enzymatic outputs.

These studies presented herein have demonstrated that stable co-cultures of two engineerable microbes, K. rhaeticus and S. cerevisiae can be recreated in the lab. By screening various co-culture conditions, these studies uncovered an interaction between K. rhaeticus and S. cerevisiae, resembling a commensal symbiotic interaction, in which the presence of S. cerevisiae promotes the growth of K. rhaeticus on sucrose medium. Exploiting this interaction, these studies developed and characterized a standard protocol that enabled reproducible co-culture of K. rhaeticus with S. cerevisiae and self-assembly of a BC-based biological material. There is growing interest in the field of synthetic ecology, which aims to construct and understand artificial communities of microbes. This co-culture system therefore is not only of interest for the development of biological ELMs but may also represent an interesting model system for synthetic ecology, in which further microbe-microbe interactions could be investigated and engineered.

Using this co-culture method, these studies demonstrated how the wealth of existing S. cerevisiae synthetic biology tools can be leveraged to program functional biological properties into grown BC materials. Firstly, these studies showed that a number of enzymes can be secreted from S. cerevisiae, become incorporated into the BC material and, therefore, functionalize the material. In addition, these studies found that functionalized BC materials can be dried and later rehydrated, retaining catalytic activity. The functionalized BC materials that were generated could be applied to the degradation of β-lactam antibiotics or estrogen hormones present in wastewater streams, both of which are environmental pollutants. Further, this approach is highly adaptable—numerous other protein targets could be secreted from S. cerevisiae to add various biological properties to the material. However, the feasibility of the approach strongly depends on the survivability of the enzymes in the growth media (pH 5-6, salt composition) or after sterilization procedures. Also, yeast might not be able to secrete every enzyme as efficiently. Given this modular approach it is possible to design, engineer and grow various yeast strains in co-cultures and screen the obtained ELM with high throughput methods.

Secondly, these studies showed how S. cerevisiae biosensor strains can be incorporated within the BC material to modify the bulk mechanical properties of BC as well as create living materials able to sense and respond to changes in their environment. The ability of cells to sense-and-respond to environmental stimuli underlies a number of interesting properties of natural biological materials, including autonomous patterning and dynamic, responsive physical properties. Once again, since this approach is highly-adaptable, numerous other S. cerevisiae biosensor strains able to detect pathogens⁴¹, environmental pollutants⁴², biomarkers⁴⁴ and so on, could be used in conjunction with the co-culture method.

So far, BC serves as a storage and protection shell for yeast and enzymes as this approach, focused mainly on engineering and exchanging S. cerevisiae. However, morphology and properties of the BC scaffold are also a target through genetic engineering of K. rhaeticus, as it is genetically tractable and more tools are being developed.

In summary, the co-culture approach to grow biofunctional ELMs combines the advantage of high yield BC production up to gram per liter quantities and its versatile modification and functionalization possibilities. Past and future advances in synthetic biology related yeast strain modifications can be easily incorporated and used to sense, degrade or assemble surrounding materials. This modular approach might even allow to tackle more complex tasks by relying on more than one engineered yeast strain.

As described herein, co-cultures of the bacterial cellulose-expressing bacteria cell, and a synthetic biology host organism are provided. In some embodiments, any type of bacterial cell that expresses bacterial cellulose can be used as described herein. In some embodiments, the bacterial cell is a Komagataeibacter cell or a Gluconacetobacter cell. In one exemplary embodiment, the bacterial call is K. rhaeticus.

In some embodiments, the synthetic biology host organism is a prokaryotic cell or a eukaryotic cell. One exemplary synthetic biology host organism is S. cerevisiae. In some embodiments the synthetic biology host organism is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In other embodiments, the cell is a fungal cell such as a yeast cell, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. Preferably the yeast strain is a S. cerevisiae strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

As described herein, a co-culture approach can be used to grow biofunctional ELMs that provide high yield BC production and functionalized materials. High yield BC production can be gram per liter quantities, such as at least 1, 2, 3, 4, 5, 6, 7, 8, 9, or 10 grams per liter, or in excess of 10 grams per liter.

Co-cultures are considered stable when neither of the two cell types outgrows the other, which can be assessed by the stability of cell numbers or cell densities in the co-culture over time, i.e., by comparing these values in at least two cycles of passage. The stability of the co-culture also can be assessed by stability of pellicle yield over time, i.e., by comparing the pellicle yield (e.g., by weight) in at least two cycles of passage. Such parameters are considered to remain stable (relatively constant) if they vary by no more than 25%, preferably no more than 20%, more preferably no more than 15%, more preferably no more than 10%, still more preferably no more than 5%. To determine stability of cell counts, they are determined in the part of the co-culture in which they are stable for each of the cell types. For example, for K. rhaeticus, cell counts were found to be consistent in the pellicle layer, where the majority of cells were detected, but more variable in the liquid layer. For S. cerevisiae, cell counts were found to be consistent in the liquid layer of the co-culture.

The co-cultures disclosed herein are stable over many cycles of passage, such as more than 10, more than 15, more than 20, more than 25, more than 30, more than 35, more than 40, more than 45, more than 50, more than 60, more than 70, more than 80, more than 90, or, more than 100 cycles.

In some embodiments, one or more of the genes associated with the products and methods disclosed herein is expressed in a recombinant expression vector, including genetic circuits that can be used to control expression of one or more output proteins of the synthetic biology host organism. As used herein, the terms “protein” and “polypeptide” are used interchangeably and thus the term polypeptide may be used to refer to a full-length polypeptide and may also be used to refer to a fragment of a full-length polypeptide.

Examples of such proteins expressed by synthetic biology host organisms in a co-culture include enzymes that can be used to provide various functionalities to the bacterial cellulose produced by the bacteria in the co-culture or to modify the physical properties of the bacterial cellulose produced by the bacteria in the co-culture. Examples of such enzymes include β-lactam hydrolyzing enzymes, such as β-lactamases; alpha-galactosidases, such as S. cerevisiae Mel1; laccases, such as laccases from Myceliophthora thermophila (MtLcc1) or from Coriolopsis troggi (CtLcc1); lactases; glucose isomerases; lipases; cellulases; cellobiohydrolases, such as CBH1 and CBH2; endoglucanases, such as EGL2; 13-glucosidases, such as BGL1; and lytic polysaccharide monooxygenases, such as LPMO. Other proteins expressed by synthetic biology host organisms can include G-protein coupled receptors (GPCRs); optical dimerizer proteins, such as the CRY2/CIB transcription system; and detectable proteins, such as fluorescent proteins and colorimetric proteins. Proteins that are homologous to the above enzymes and proteins also can be used.

In some embodiments, the proteins such as enzymes are linked to a cellulose binding protein, such as a cellulose-binding domain (CBD) to provide a specific, stable binding interaction between the enzyme and cellulose. In some embodiments, the CBD is fused to the C-terminus of the protein, such as an enzyme. In some embodiments, the CBD is CBDcex, the 112 amino acid region from the C-terminus of the Cex exoglucanase from Cellulomonas fimi.

To secrete such enzymes into the co-culture media for binding to bacterial cellulose and incorporation into the pellicle, variants of each protein can be made that include a suitable secretion signal peptide such that the protein is exported from the synthetic biology host cell. For example, if a native secretion signal peptide is present in the protein, it can be used and tested for suitability in the synthetic biology host cell. Alternatively, a secretion signal peptide from the synthetic biology host cell itself can be used, such as the S. cerevisiae MFα signal peptide.

As used herein with respect to cells, cultures, co-cultures, pellicles, polypeptides, proteins, or fragments thereof, “isolated” means separated from its native environment and present in sufficient quantity to permit its identification or use. Isolated, when referring to a protein or polypeptide, means, for example: (i) selectively produced by expression cloning or (ii) purified such as by chromatography or electrophoresis. Isolated proteins or polypeptides may be, but need not be, substantially pure. The term “substantially pure” means that the proteins or polypeptides are essentially free of other substances with which they may be found in production, nature, or in vivo systems to an extent practical and appropriate for their intended use. Substantially pure polypeptides may be obtained naturally or produced using methods described herein and may be purified with techniques well known in the art. Because an isolated protein, for example, may be admixed with other components in a preparation, the protein may comprise only a small percentage by weight of the preparation. The protein is nonetheless isolated in that it has been separated from the substances with which it may be associated in living systems, i.e. isolated from other proteins.

As used herein, a “vector” may be any of a number of nucleic acids into which a desired sequence or sequences may be inserted by restriction and ligation for transport between different genetic environments or for expression in a host cell. Vectors are typically composed of DNA, although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably joined” (or “operably linked”) when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors used herein may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (or RNA). That heterologous DNA (or RNA) is placed under operable control of transcriptional elements to permit the expression of the heterologous DNA (or RNA) in the host cell. For example, heterologous expression of genes associated with the disclosed methods and products, such as for production of enzymes in a synthetic biology host cell such as S. cerevisiae, is demonstrated herein.

A nucleic acid molecule as used in the products and methods disclosed herein can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule may also be accomplished by integrating the nucleic acid molecule into the genome.

In some embodiments one or more genes used in the products and methods disclosed herein is expressed recombinantly in a bacterial cell. Bacterial cells can be cultured in media of any type (rich or minimal) and any composition. As would be understood by one of ordinary skill in the art, a variety of types of media can be compatible with the products and methods disclosed herein. However, as shown herein, certain types of media or components of media (e.g., carbon source) provide superior results for the products and methods disclosed herein. The selected medium can be supplemented with various additional components. Some non-limiting examples of supplemental components include glucose or other carbon sources (such as described elsewhere herein), antibiotics, IPTG for gene induction, ATCC Trace Mineral Supplement, and glycolate. Similarly, other aspects of the medium, and growth conditions of the cells used in the products and methods disclosed herein may be optimized through routine experimentation. In some embodiments, factors such as choice of media, media supplements, and temperature can influence growth of organisms in the co-cultures described herein, or production levels of bacterial cellulose or of enzymes or other proteins expressed in organisms in the co-cultures. In some embodiments the concentration and amount of a supplemental component may be optimized. In some embodiments, how often the media is supplemented with one or more supplemental components, and the amount of time that the organisms are co-cultured is optimized.

The term “linked” or “linkage” refers to an association of two entities, for example, of two molecules such as two proteins, or a protein and a reactive handle, or a protein and an agent, e.g., a detectable label. The association can be, for example, via a direct or indirect (e.g., via a linker) covalent linkage or via non-covalent interactions. In some embodiments, the association is covalent. In some embodiments, two molecules are linked via a linker connecting both molecules. For example, in some embodiments where two proteins are linked to each other to form a fusion protein, the two proteins may be linked via a polypeptide linker, e.g., an amino acid sequence connecting the C-terminus of one protein to the N-terminus of the other protein.

The term “detectable label” refers to a moiety that has at least one element, isotope, or functional group incorporated into the moiety which enables detection of the molecule, e.g., a protein or peptide, or other entity, to which the label is attached. Labels can be directly attached or can be attached via a linker. It will be appreciated that the label may be attached to or incorporated into a molecule, for example, a protein, polypeptide, or other entity, at any position. In general, a detectable label can fall into any one (or more) of five classes: I) a label which contains isotopic moieties, which may be radioactive or heavy isotopes, including, but not limited to, ²H, ³H, ¹³C, ¹⁴C, ¹⁵N, ¹⁸F, ³¹F, ³²F, ³⁵S, ⁶⁷Ga, ⁷⁶Br, ⁹⁹mTc (T-⁹⁹m) ¹¹¹In, ¹²³I, ¹²⁵I, ¹³¹I, ¹⁵³Gd, ¹⁶⁹Yb and ¹⁸⁶Re; II) a label which contains an immune moiety, which may be antibodies or antigens, which may be bound to enzymes (e.g., such as horseradish peroxidase); III) a label which is a colored, luminescent, phosphorescent, or fluorescent moieties (e.g., such as the fluorescent label fluorescein-isothiocyanate (FITC); IV) a label which has one or more photo affinity moieties; and V) a label which is a ligand for one or more known binding partners (e.g., biotin-streptavidin, FK506-FKBP). In certain embodiments, a label comprises a radioactive isotope, preferably an isotope which emits detectable particles, such as beta particles. In certain embodiments, the label comprises a fluorescent moiety. In certain embodiments, the label is the fluorescent label fluorescein-isothiocyanate (FITC). In certain embodiments, the label comprises a ligand moiety with one or more known binding partners. In certain embodiments, the label comprises biotin, which may be detected using a streptavidin conjugate (e.g., fluorescent streptavidin conjugates such as Streptavidin ALEXA FLUOR® 568 conjugate (SA-568) and Streptavidin ALEXA FLUOR® 800 conjugate (SA-800), Invitrogen). In some embodiments, a label is a fluorescent polypeptide (e.g., GFP or a derivative thereof such as enhanced GFP (EGFP)) or a luciferase (e.g., a firefly, Renilla, or Gaussia luciferase). It will be appreciated that, in certain embodiments, a label may react with a suitable substrate (e.g., a luciferin) to generate a detectable signal. Non-limiting examples of fluorescent proteins include GFP and derivatives thereof, proteins comprising fluorophores that emit light of different colors such as red, yellow, and cyan fluorescent proteins. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols Methods of biochemical analysis, v. 47 Wiley-Interscience, Hoboken, N.J., 2006; and Chudakov, D M, et al., Physiol Rev. 90(3):1103-63, 2010, for discussion of GFP and numerous other fluorescent or luminescent proteins. In some embodiments, a label comprises a dark quencher, e.g., a substance that absorbs excitation energy from a fluorophore and dissipates the energy as heat.

The term “homologous,” as used herein, is an art-understood term that refers to nucleic acids or polypeptides that are highly related at the level of nucleotide or amino acid sequence. Nucleic acids or polypeptides that are homologous to each other are termed “homologs” or “homologues.” Homology between two sequences can be determined by sequence alignment methods known to those of skill in the art. Two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids, or over the full sequence of one or both of the molecules being compared. The “percent identity” of two nucleic acid or two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleic acid or protein searches can be performed with the XBLAST program, score=50, wordlength=3 to obtain sequences homologous to the nucleic acid or protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

The products and methods disclosed herein are further illustrated by the following Examples, which in no way should be construed as further limiting. It should be understood that these Examples, while indicating preferred embodiments, are given by way of illustration only. From the above discussion and these Examples, one skilled in the art can ascertain the essential characteristics of the products and methods disclosed herein, and without departing from the spirit and scope thereof, can make various changes and modifications to adapt the products and methods disclosed herein to various uses and conditions.

The entire contents of all of the references (including literature references, issued patents, published patent applications, and co-pending patent applications) cited throughout this application are hereby expressly incorporated by reference, particularly for the teachings referenced herein.

### EXAMPLES

### Example 1. Materials and Methods

Strains, constructs and DNA assembly: Strains and DNA constructs used in this study include Kr, BY4741, yCelMix, yCBH1, yCBH2, yBGL1, yEGL2, yLPMO, yXTH3, yTT-GFP, yRR-GFP, yTR-GFP, yRT-GFP, yNC, yNS, yNSΔSED1. The plasmids constructed in this study were constructed using standard cloning techniques. Oligonucleotides were obtained from IDT. Restriction endonucleases, Phusion-HF DNA polymerase and T7 DNA ligase were obtained from NEB. Unless stated, the plasmids were transformed into E. coli turbo (NEB) for amplification and verification before transforming into S. cerevisiae for protein expression and secretion. The constructs were verified by restriction enzyme digestion and Sanger sequencing (Source Bioscience).

S. cerevisiae constructs for strains yCG01, yCG02, yCG04 and yCG05 were cloned using the yeast toolkit (YTK) system developed by the Dueber lab²⁷. The YTK system uses Golden Gate assembly to combine pre-assembled, defined parts into single gene cassettes and multi-gene cassettes. The final positions of pre-assembled parts within constructs are determined by the sequences of 4 bp overhangs created by digestion with type IIS restriction enzymes (BsaI or BsmBI). Users can therefore pick and choose from pre-assembled promoter, terminator and protein-coding parts to create expression cassettes. The combination of parts used to create strains yCG01, yCG02, yCG04 and yCG05 were cloned into a pre-assembled backbone plasmid, pYTK096, containing genetic elements enabling cloning in E. coli and later integrative transformation into the URA3 locus in S. cerevisiae. Type 2, 3 and 4 parts were cloned into the pre-assembled backbone. To create more complex fusion proteins, additional subparts were used (e.g., 3a and 3b parts). New parts were codon optimized for S. cerevisiae expression, synthesized commercially by GeneArt or IDT and cloned into the YTK system entry vector, pYTK001, for storage and verification. The other parts were taken from the YTK. Golden gate assembly reactions were performed as described in Lee et al.²⁷. Other strains, including Sc GFP, yWS890 and yGPY093, were similarly constructed using the YTK system and kindly provided by the Ellis lab.

Culture conditions and media: Yeast extract peptone dextrose (YPD) and yeast extract peptone sucrose (YPS) media were prepared with 10 g/L yeast extract, 20 g/L peptone and 20 g/L glucose or sucrose. Synthetic complete (SC) dropout media were prepared with 1.4 g/L yeast synthetic dropout medium supplements, 6.8 g/L yeast nitrogen base without amino acids and 20 g/L glucose. Depending on the specified selection, SC media were supplemented with stock solutions of one or more of uracil (final concentration 2 g/L), tryptophan (final concentration 50 mg/L), histidine (final concentration 50 mg/L) and leucine (final concentration 0.1 g/L). Hestrin-Schramm (HS) media were prepared with 5 g/L yeast extract, 5 g/L peptone, 2.7 g/L Na₂HPO₄, 1.5 g/L citric acid and 20 g/L glucose or sucrose. Where needed, media were supplemented with 20 g/L bacteriological agar. Partway through this study, a switch was made between sources of peptone for co-culture medium preparation from peptone from casein, to peptone from soybean. It was noted that peptone from soybean resulted in higher and more consistent pellicle yields.

E. coli was grown in LB medium at 37° C., supplemented with appropriate antibiotics at the following concentrations: chloramphenicol 34 μg/mL, kanamycin 50 μg/mL. For biomass accumulation, K. rhaeticus was grown at 30° C. in yeast extract peptone dextrose (YPD) medium supplemented with 34 μg/mL chloramphenicol and 1% (v/v) cellulase from T. reseii (Sigma Aldrich, C2730). It was found that the growth of K. rhaeticus liquid cultures was significantly more reliable when inoculated from glycerol stock, rather than from colonies. Therefore, unless otherwise indicated, the K. rhaeticus cultures were inoculated from glycerol stocks. S. cerevisiae was grown at 30° C. in rich YPD medium or selective, SC medium lacking the appropriate supplements, each supplemented with 50 μg/mL kanamycin.

Co-culture condition screen: Triplicate samples of K. rhaeticus Kr RFP were inoculated from glycerol stocks into 5 mL YPD medium supplemented with cellulase (1% v/v) and grown in shaking conditions for 3 days. Triplicate samples of S. cerevisiae Sc GFP were inoculated from plates into 5 mL YPD medium and grown in shaking conditions for 24 hours. To prepare screens, K. rhaeticus and S. cerevisiae were inoculated into 2 mL volumes of YPD, YPS, HS-glucose or HS-sucrose media in 24-well cell culture plates. K. rhaeticus cultures were diluted 1/50 into fresh media. S. cerevisiae cultures were inoculated over a range of dilutions: 1/100, 1/1000, 1/10,000, 1/100,000 and 1/1,000,000. To enable pellicle formation, plates were incubated for 4 days under static conditions at 30° C. After 4 days of incubation, cultures were photographed under identical conditions. Where present, pellicle layers were removed from the culture surface and photographed.

Standard co-culture protocol: Triplicate samples of K. rhaeticus were inoculated from glycerol stocks into 5 mL YPD medium supplemented with cellulase (1% v/v) and grown in shaking conditions for 3 days. Triplicate samples of S. cerevisiae were inoculated from plates into 5 mL YPD medium and grown in shaking conditions for 24 hours. To enable inoculation of co-cultures with equivalent cell densities of different samples, OD₆₀₀ measurements were made and used to normalize pre-culture densities. K. rhaeticus pre-cultures were centrifuged at 3220×g for 10 min and cell pellets resuspended in sufficient volume of YPS medium to result in a final OD₆₀₀ of 2.5. S. cerevisiae pre-cultures were diluted in YPS medium to a final OD₆₀₀ of 0.01. To prepare final co-cultures, resuspended K. rhaeticus samples were diluted 1/50 and pre-diluted S. cerevisiae samples were diluted 1/100 into fresh YPS medium. In instances where strains were inoculated into various different final media, K. rhaeticus pellets were resuspended in PBS buffer and S. cerevisiae cultures were pre-diluted in PBS buffer. To prepare OptiPrep-containing co-cultures, OptiPrep (D1556, Sigma-Aldrich) was added to YPS media to a final concentration of 45% (v/v). Co-cultures were grown in 55 mm petri dishes (15 mL) or 12 well cell culture plates (4 mL). Co-cultures were incubated for 3 days at 30° C. under static conditions. For even pellicle formation, culture vessels should not be disturbed during the incubation period.

Determining BC pellicle yields: To determine the yields of BC pellicles, pellicle layers were removed from the surfaces of cultures and dried using the ‘sandwich method’. Here, pellicles were sandwiched between two sheets of greaseproof paper and then further sandwiched between multiple sheets of absorbent paper and finally placed under a heavy weighted object. After 24 hours, fresh sheets of absorbent paper were added and pellicles were then left for an additional 24 hours. Pellicles dried in this way were then weighed to determine pellicle yields. Pellicles were not treated with NaOH to lyse and remove cells embedded within the BC matrix.

This method was used to follow the yields of pellicle formation over time. Here, multiple co-cultures were prepared in triplicate using Sc GFP and Kr RFP strains and the standard co-culture procedure. Co-cultures were grown in 12 well plate format. At indicated time points, pellicle layers were removed to be dried and weighed.

This method was also used to compare the pellicle yield between K. rhaeticus mono-culture and co-culture with S. cerevisiae. Here, mono-cultures of K. rhaeticus (Kr RFP) were prepared in YPD medium and in co-culture with S. cerevisiae (Sc GFP) in YPS medium, using the standard co-culture protocol. After 3 days of incubation at 30° C., pellicle layers were removed to be dried and weighed.

Co-culture passage: To test whether co-cultures could be passaged, initial co-cultures between S. cerevisiae (Sc GFP) and K. rhaeticus (Kr RFP) were prepared in triplicate in 15 mL YPS cultures using the standard co-culture protocol. After 3 days incubation at 30° C., photographs were taken of the resultant cultures. To initiate new rounds of growth, pellicle layers were removed and the liquid below mixed by aspiration and diluted 1/100 into fresh samples of 15 mL YPS. This process was repeated over 16 rounds.

To confirm that the initial strain of GFP-expressing S. cerevisiae (Sc GFP) was maintained during passage, samples were plated at the end of each round. Samples from both the liquid below the pellicle and the pellicle layer itself were plated at various dilutions onto YPD-kanamycin plates. To enable plating, pellicles were digested by shaking gently for 16 hours at 4° C. in 15 mL of PBS buffer with 2% (v/v) cellulase from T. reseii (Sigma Aldrich, C2730). After 48 hours of incubation at 30° C., plates were imaged for GFP fluorescence. Dilutions were selected which enabled visualization of single colonies. Initially plates were imaged using a Fujifilm FLA-5000 Fluorescent Image Analyzer. However, due to equipment malfunction, later plates were photographed under a transluminator.

Determining cell distribution in co-cultures: Cell distributions were determined by plating samples of cells onto solid media and counting the resultant colonies. Pellicle samples were first rinsed by inverting ten times in 15 mL PBS and then digested by shaking gently for 16 hours at 4° C. in 15 mL of PBS buffer with 2% (v/v) cellulase from T. reseii (Sigma Aldrich, C2730). Samples were diluted at various levels into PBS. For S. cerevisiae cell counts, samples were plated onto YPD-kanamycin media. For K. rhaeticus cell counts, samples were plated onto SC media lacking the four supplements essential for S. cerevisiae growth (histidine, leucine, tryptophan and uracil). In these instances, Kr RFP and Sc GFP strains were used. Therefore, to ensure the colonies counted were the target strains, plates were scanned for fluorescence using a Fujifilm FLA-5000 Fluorescent Image Analyzer. Plate cell counts were used to calculate the original colony forming units (cfu) per unit volume for liquid samples. However, since the exact volumes of pellicle were not measured prior to degradation, it was not possible to calculate the exact cell counts in cfu per unit volume. To enable a rough approximation of the cell counts per unit volume, pellicle volumes were estimated at fixed levels and these values were used to calculate estimated cfu per unit volume. For 15 mL cultures, pellicle volumes were estimated at 4 mL and for 4 mL cultures in 12 well plates pellicle volumes were estimated at 1 mL.

To compare cell counts from mono-cultures and co-cultures of K. rhaeticus and S. cerevisiae, pre-cultures of K. rhaeticus Kr RFP were pelleted and resuspended in PBS buffer and pre-cultures of S. cerevisiae Sc GFP were diluted in PBS buffer, according to the standard co-culture procedure. Various co-cultures and mono-cultures were then prepared in different media in 15 mL volumes. After 3 days incubation at 30° C., pellicle and liquid samples were prepared, diluted and plated for cell counts.

To determine the reproducibility of co-culture cell counts, co-cultures were prepared according to the standard co-culture protocol in 15 mL cultures on three separate occasions. After 3 days incubation at 30° C., pellicle and liquid samples were prepared, diluted and plated for cell counts.

To determine cell counts in BC balls, balls were degraded by gently mixing for 16 hours at 4° C. in 1 mL of PBS buffer with 2% (v/v) cellulase from T. reseii (Sigma Aldrich, C2730). Degraded ball samples were diluted and plated for cell counts as above. To estimate the cell counts of S. cerevisiae in cfu per unit volume, a ball diameter of 3 mm was assumed.

Fluorescence microscopy: Images of pellicles were prepared using a 20× objective lens mounted on a Nikon Eclipse Ti inverted microscope. Slices of pellicle were mounted on slides with the bottom face of the pellicle facing downwards. To visualize samples, a phase filter (Ph1) was used to enhance contrast. GFP fluorescence images were taken using 480 nm excitation and 535 nm emission wavelengths. The NIS-elements microscope imaging software was used for initial image capture and ImageJ was used for downstream image analysis and stacking of GFP and brightfield images.

Invertase supplementation experiment: Co-cultures and K. rhaeticus Kr RFP mono-cultures were prepared in YPS medium according to the standard co-culture procedure. Recombinant, purified S. cerevisiae invertase (Sigma-Aldrich, 19274) was resuspended in 100 mM citrate buffer, pH 4.5 to create a stock solution at a final concentration of 5 U/μL. This stock solution was diluted into YPS medium for a range of final invertase concentrations: 50 mU/mL (10⁻²), 5 mU/mL (10⁻³), 0.5 mU/mL (10⁴), 50 μU/mL (10⁻⁵). After 3 days growth at 30° C., cultures and, where present, pellicles were imaged.

Supernatant nitrocefin assay: For culture supernatant assays, WT BY4741, yCG04 and yCG05 S. cerevisiae strains were grown in triplicate overnight in YPD liquid medium with shaking. As used in the studies presented herein, the WT BY4741 strain used was MATa his3Δ1 leu2Δ0 met15Δ0 ura3Δ0, from the Dharmacon yeast collection. After 16 hours growth, liquid cultures were back-diluted to final OD₆₀₀=0.01 in 5 mL fresh YPS medium and grown for 24 hours with shaking. The resultant cultures were centrifuged at 3220×g for 10 min and the supernatant fractions harvested. Supernatant samples were pipetted in 50 μL volumes into the wells of a 96 well plate. The colorimetric substrate, nitrocefin (484400, Merck-Millipore), was resuspended in DMSO to create a 10 mg/mL working stock. This stock was diluted to 50 μg/mL in nitrocefin assay buffer (50 mM sodium phosphate, 1 mM EDTA, pH 7.4). To start the reaction, 50 μL of nitrocefin at 50 μg/mL was added to each of the samples simultaneously and the absorbance at 490 nm was measured over time. Active β-lactamase converts nitrocefin to a red substrate, increasing the absorbance of light at 490 nm. Therefore, to calculate the relative β-lactamase activity in samples, the rate of change in the absorbance of light at 490 nm was determined. Specifically, the product formation rates were calculated from the gradient over the linear region of a graph plotting fluorescence AU over time.

Pellicle nitrocefin assays: For initial pellicle assays, WT BY4741, yCG04 and yCG05 S. cerevisiae strains were co-cultured with K. rhaeticus (Kr RFP) in triplicate, according to the standard co-culture protocol. Following 3 days growth, pellicles were removed and washed in 15 mL PBS buffer for 30 min with shaking at 150 rpm. Square pieces of pellicle, measuring 5 mm×5 mm, were then cut using a scalpel. The remainder of the pellicle was dried using the sandwich method. Once dried, pellicles were again cut to produce 5 mm×5 mm pieces. Dried pellicle pieces were rehydrated by adding 25 μL of PBS buffer and incubating for 30 min. Assays for both wet and dried samples were run by adding 10 μL of nitrocefin, diluted to 1 mg/mL in PBS buffer, to each of the pellicle pieces simultaneously. Initial assays were performed at room temperature. Photographs were taken of pellicles over the course of 35 min to follow the color change. To provide a quantitative measure of color change, the ImageJ (NIH) image analysis software was used. Images were first split into individual color channels. Since yellow-to-red color change is caused by an increase in the absorbance of green light wavelengths, the green channel was selected. To quantify the yellow-to-red color change, the green channel intensity was then measured from greyscale-inverted images of pellicle slices over time. Since preliminary results showed that WT pellicles exhibited no color change, the signal from WT pellicles was used as a baseline value to correct for background levels of green channel intensity.

To determine absolute levels of β-lactamase activity in wet and dried pellicles, a similar protocol was used to create standard curves. Standard curves were prepared using a commercial E. coli β-lactamase enzyme (ENZ-351, ProSpec). First, pellicles grown with WT BY4741 S. cerevisiae were washed in nitrocefin assay buffer (50 mM sodium phosphate, 1 mM EDTA, pH 7.4). Pieces measuring 5 mm×5 mm were cut and weighed to enable determination of the approximate volume of liquid within the pellicle. The remainder of the pellicles were dried using the sandwich method. Once dried, 5 mm×5 mm pieces of pellicle were cut for dried pellicle standard curves. Dried pellicle pieces were rehydrated by adding 20 μL of nitrocefin assay buffer. Pre-diluted standard β-lactamase samples were then added to pellicle pieces in 5 μL volumes and allowed to diffuse throughout the BC for 30 min. To initiate the reaction, 5 μL aliquots of nitrocefin, diluted to 2 mg/mL in nitrocefin assay buffer, were added to each of the pellicle pieces simultaneously. Samples were incubated at 25° C. and photographs taken over the course of the reaction. Again, ImageJ was used to quantify the yellow-to-red color change at given time points. Time points were chosen to maximize the dynamic range, without reaching saturation. For wet pellicles, it was necessary to use the measured weight of pellicle slices to determine the actual final concentration of the standard β-lactamase. Standard curves using fresh wet pellicles, dried pellicles and dried pellicles stored for 1 month or 6 months at room temperature were prepared according to this method. For long-term storage, dried pellicles were stored in petri dishes at room temperature and protected from light.

Alongside standard curves, pellicles grown with yCG05 S. cerevisiae were analyzed using an identical protocol. To enable cross comparison with standard curves, negative samples (pellicles from co-cultures with WT S. cerevisiae) and positive samples (pellicles from co-cultures with WT S. cerevisiae to which a known amount of β-lactamase standard had been added) were run with samples. For samples to which no standard β-lactamase was added, 5 μL of nitrocefin assay buffer was added to maintain equal final liquid volumes. Photographs taken at identical time points were then used with standard curves to calculate absolute values of β-lactamase activity. Again, ImageJ was used to quantify the yellow-to-red color change. For wet pellicles, it was necessary to use the measured weight of pellicle slices to determine the actual final concentration of enzyme. Again, fresh wet pellicles, dried pellicles and dried pellicles stored for 1 month at room temperature were assayed according to this method.

β-lactamase activity retention assay: To determine the retention of β-lactamase within BC following multiple rounds of washes, nitrocefin assays were performed. Pieces measuring 5 mm×5 mm were cut from dried pellicles grown with yCG04 and yCG05. The pellicle pieces were rehydrated by incubating in 1 mL of PBS buffer. Pieces were subjected to a variable number of wash steps, where pellicle pieces were incubated in 4 mL PBS buffer at 25° C. and 150 rpm for 30 min. After washing, pellicles were assayed for β-lactamase activity. Negative samples (pellicles from co-cultures with WT S. cerevisiae) and positive samples (pellicles from co-cultures with WT S. cerevisiae to which a known amount of β-lactamase standard had been added) were run alongside the samples. For samples to which no standard β-lactamase was added, 5 μL of PBS buffer was added to maintain equal final liquid volumes. As before, assays were initiated by adding 5 μL of nitrocefin, diluted to 2 mg/mL in PBS buffer, to each of the pellicle pieces simultaneously. Since the number of samples that can be run in parallel is limited, samples were run in batches based on the number of washes. Again, ImageJ was used to quantify the yellow-to-red color change at given time points. To enable cross-comparison between different assay runs, negative samples were used to subtract background signals and positive samples were used to normalize signals. To ensure that yellow-to-red color change values were within a range in which there is a linear relationship between β-lactamase activity and the yellow-to-red color change signal, a standard curve was run. The standard curve (r²=0.9571) confirmed that detected yellow-to-red color change values fell within the linear range.

X-α-gal α-galactosidase assays: A stock solution of X-α-galactosidase (Sigma-Aldrich, 16555) was prepared in DMSO at a concentration of 40 mg/mL. For plate assays, 100 μL of X-α-gal were spread on plates prior to cell plating and images taken after 3 days growth at 30° C. For pellicle assays, pellicles grown with K. rhaeticus Kr RFP were harvested after 3 days growth following the standard co-culture procedure. Pellicles were then washed in 15 mL 100 mM citrate buffer, pH 4.5 for 30 min with shaking at 150 rpm. Square pieces of pellicle, measuring 5 mm×5 mm, were then cut using a scalpel. The remainder of the pellicle was dried using the sandwich method. Once dried, pellicles were again cut to produce 5 mm×5 mm pieces. Dried pellicle pieces were rehydrated by adding 25 μL of 100 mM citrate buffer, pH 4.5 and incubating for 30 min. Assays for both wet and dried samples were run by adding 2.5 μL of X-α-gal stock solution and incubating at 25° C. Images were taken over the course of several hours.

ABTS laccase activity assays: Stock solutions were prepared of 2,2′-azino-bis(3-ethylbenzothiazoline-6-sulphonic acid) (ABTS) (Sigma-Aldrich, A1888) at a final concentration of 0.1M and copper sulphate at a final concentration of 1M. Laccases are copper-containing enzymes, requiring supplementation of copper for culture and assay conditions. For plate assays, 125 μL of 0.1M ABTS and 25 μL of 1M CuSO₄ were spread on plates prior to cell plating and images taken after 3 days growth at 30° C. For pellicle assays, pellicles grown with K. rhaeticus Kr RFP were harvested after 3 days growth following the standard co-culture procedure. The only modification was the addition of 1 mM CuSO₄ to the culture medium of both S. cerevisiae pre-cultures and co-cultures. Pellicles were then washed in 15 mL 100 mM citrate buffer, 1 mM CuSO₄, pH 4.5 for 30 min with shaking at 150 rpm. Square pieces of pellicle, measuring 5 mm×5 mm, were then cut using a scalpel. The remainder of the pellicle was dried using the sandwich method. Once dried, pellicles were again cut to produce 5 mm×5 mm pieces. Dried pellicle pieces were rehydrated by adding 25 μL of 100 mM citrate buffer, 1 mM CuSO₄, pH 4.5 and incubating for 30 min. Assays for both wet and dried samples were run by adding 5 μL of ABTS stock solution and incubating at 25° C. Images were taken over the course of several hours.

Assaying GFP secretion into supernatant and pellicles: As preliminary test of ability of yCG01 and yCG02 to secrete GFP, individual colonies were grown in 5 mL YPD medium for 48 hours. As a negative control strain, non-fluorescent yCG04 was used. After 48 hours growth, cultures were centrifuged at 3220×g for 10 min and supernatant samples imaged for fluorescence under a transluminator.

To test whether S. cerevisiae could secrete detectable levels of GFP into BC materials, co-cultures were prepared in triplicate according to the standard co-culture protocol using WT BY4741, yCG01 and yCG02 strains. Co-cultures were grown in 4 mL volumes in 12 well plates. Since GFP secretion yields were anticipated to be low, co-cultures were allowed to grow for either 7 days or 14 days before imaging. After incubation, pellicles were washed by incubating for 30 min in 15 mL PBS buffer. Washed pellicles were then imaged for GFP using Fujifilm FLA-5000 Fluorescent Image Analyzer. Images were analyzed and modified for presentation using ImageJ (NIH). Specifically, brightness and contrast were adjusted, equally for all samples, to the point at which the background fluorescence of pellicles grown with WT S. cerevisiae was just visible.

Scanning electron microscopy (SEM): Pellicles were grown for 3 days following the co-culture procedure and washed with deionized water 3 times (shaking at 70 rpm at 4° C. for 12 hours per wash) to remove residue YPS or OptiPrep. Washed pellicles were then free-dried with a lyophilizer for at least 48 hours before coated with a gold sputter. Images were taken with a JEOL 6010LA benchtop scanning electron microscope.

Brunauer-Emmett-Teller (BET) surface area analysis: Free-dried pellicles were cut into 5 mm×5 mm piece and placed in sample tube for 1 hour degas at 423 K using a Micromeritics (Atlanta, Ga.) ASAP 2020 analyzer. BET surface area and pore size were then determined with N₂ adsorption at 77 K using Brunauer-Emmett-Teller and Barrett-Joyner-Halenda analyses on the same machine.

Preparing and assaying sense-and-response pellicles: In GPY093 transcription from the BED-inducible promoter is controlled by a synthetic transcription factor (Z3EV) consisting of three domains: the Zif268 DNA-binding domain, the human estrogen receptor (hER) ligand binding domain, and the transcriptional activation domain of viral protein 16 (VP16^{AD})⁴⁵. When present, β-estadiol binds to the hER ligand binding domain of Z3EV, releasing it from its basal sequestration in the cytosol and enabling it to translocate into the nucleus. Once in the nucleus, the Zif268 domain binds cognate DNA sequences in engineered promoters and the VP16^{AD} domain activates transcription of downstream genes. As a preliminary test of S. cerevisiae sense-and-response in BC pellicles, co-cultures were prepared in triplicate according to the standard co-culture protocol using WT BY4741 and GPY093 strains. Co-cultures were inoculated into 4 mL YPS-OptiPrep medium in 12 well cell culture plates. After 3 days of growth, pellicles were removed and washed by incubating at 25° C. with shaking at 150 rpm in 15 mL PBS. Pellicles were then placed in fresh 15 mL of YPD medium in the presence or absence of 5 nM β-estradiol (E8875, Sigma-Aldrich) and incubated for 24 hours at 30° C. and 150 rpm. Large quantities of cells had ‘escaped’ from biosensor pellicles, making the medium surrounding the pellicles turbid. Therefore, to remove loosely-associated cells, pellicles were washed twice by incubating at for 30 min at 25° C. and 150 rpm in 15 mL of PBS buffer. Finally, pellicles were imaged simultaneously for GFP fluorescence under a transluminator.

Similarly, dried biosensor pellicles were prepared were prepared in triplicate according to the standard co-culture protocol using WT BY4741 and yGPY093 or WT BY4741 and yWS890 strains. Co-cultures were inoculated into 4 mL YPS-OptiPrep medium in 12 well cell culture plates. After 3 days of growth, pellicles were dried using the ‘sandwich method’. Dried pellicles were then placed in fresh 15 mL of YPD medium in the presence or absence of 5 nM β-estradiol or 50 nM S. cerevisiae α-mating factor (RP01002, GenScript) and incubated 24 hours at 30° C. To more closely match the potential use of biosensors in an on-site detection setting, pellicles were incubated without agitation in this and subsequent experiments. Static growth was chosen to more closely mimic a potential in-the-field application. Since static growth result in far less growth in the surrounding liquid, pellicles were only briefly washed by inverting ten times in 15 mL PBS buffer. Finally, pellicles were imaged side-by-side for GFP fluorescence under a transluminator. To test for stability after long-term storage, pellicles were stored for 4 months at room temperature stored in petri dishes protected from light. These pellicles were cut in half prior to induction, which was performed as above.

The BED-inducible CtLcc1-secreting strain yCG23 was initially screened for laccase induction using a plate-based ABTS assay. Transformants of yCG23 were re-streaked in triplicate on SC URA⁻ plates supplemented with 125 μL of 0.1 M ABTS and 25 μL of 1 M CuSO₄. After 3 days of incubation at 30° C. colonies were imaged. Co-cultures between K. rhaeticus Kr RFP and yCG01 or yCG23 were then prepared in triplicate in 12-well plate format, using YPS-OptiPrep medium supplemented with 1 mM CuSO₄. After 3 days growth, pellicles were harvested and were washed by incubating for 30 min at 25° C. and 150 rpm in 15 mL of 100 mM citrate buffer, 1 mM CuSO₄, pH 4.5. Pellicles were then inoculated into 15 mL of fresh YPD supplemented with 1 mM CuSO₄ in the presence or absence of 5 nM β-estradiol and incubated at 30° C. for 24 hours statically. After incubation, pellicles were washed by incubating for 30 min at 25° C. and 150 rpm in 15 mL of 100 mM citrate buffer, 1 mM CuSO₄, pH 4.5. Pellicles were then placed in a 12-well plate and 75 μL of 0.1 M ABTS added to each well to assay for laccase activity. Pellicles were incubated at 25° C. and imaged after 72 hours.

In other embodiments of the ABTS assay, the plate was sealed with breathe-easy, which should allow gas exchange, since oxygen is needed for the reaction. A faint green color was detected after 48 hours and 72 hours, at which time, the film was removed and only a few hours later the intensity of the color was much, much stronger.

Determining the viability of S. cerevisiae in dried BC pellicles: Co-cultures were prepared in triplicate according to the standard co-culture protocol using Sc GFP and Kr RFP. Co-cultures were inoculated into 4 mL YPS-OptiPrep medium in 12 well cell culture plates. Counts of viable S. cerevisiae cells within wet and dried pellicles were determined as described previously. Dried pellicles were also stored for 1 month at room temperature, and then degraded and plated onto YPD medium. Since one of the triplicate samples produced no colonies, estimated cell counts within pellicles could not be calculated. However, images of the three plates showed that viable cells were indeed recovered from the other two samples.

Total cellulase activity assay: Yeast strains BY4741 and yCelMix were grown overnight in YPS in triplicate with shaking. After 16 hours growth, liquid cultures were back-diluted to final OD₆₀₀=0.1 in 5 mL fresh YPS medium with 2 mM L-ascorbic acid (A7506, Sigma-Aldrich) and grown for 24 hours with shaking. The resultant cultures were centrifuged at 3220×g for 10 min and the supernatant fractions harvested. Supernatant samples were pipetted in 50 μL volumes into the wells of a 96 well plate. The EnzChek® Cellulase Substrate (E33953, Thermo-Fisher) was resuspended in 50% DMSO and diluted 5-fold in 100 mM sodium acetate (pH 5.0). To start the reaction, 50 μL of cellulase substrate was added to the supernatant and let incubated for 30 minutes in dark at room temperature. To build an enzyme activity standard curve, the cellulase from T. reesi (C2730, Sigma-Aldrich) was used to prepare a serial dilution in YPS medium and mixed with the substrate at 1:1 ratio. Blue fluorescence (360/460) was detected using a plate reader (Synergy H1, BioTek) after 30 minutes incubation in dark at room temperature. The data from enzyme standards was fit to an exponential model, a*exp(b*x)+c*exp(d*x) in MATLAB. This model was then used to calculate the total cellulase activity of the supernatant from yCelMix (using supernatant from BY4741 as a blank control).

Pellicle tensile test: Co-cultures were set up in 40 mL YPS+OptiPrep (plus 2 mM L-ascorbic acid) and grown in square plates (100 mm×15 mm) for 2 days at 30° C. Pellicles were then washed in deionized water 3 times (shaking at 70 rpm at 4° C. for 12 hours per wash) and dried using the sandwich method described previously but with an extended 3 days drying to ensure water removal. Dried pellicles were cut into 60 mm*10 mm stripes and their thickness were measured with a micrometer. Tensile test was performed with a Zwick mechanical tester (BTC-ExMacro 0.001, Roell) following the ASTM D882 protocol at 1 mm/min speed.

Pellicle rheology analysis: The rheological properties of washed pellicles were characterized on a rheometer (AR2000, TA Instruments) with a 25 mm ETC aluminum plate (1 mm gap). The strain sweep measurements were taken from 0.01% to 100% strain amplitude at a constant frequency of 1 rad/s while frequency sweep measurements were taken from 0.1 rad/s to 100 rad/s at a constant strain amplitude of 1%. Samples were kept fully-hydrated with deionized water at 25° C. on a Peltier thermoelectric plate.

Light-inducible circuit promoter characterization: Yeast strains were grown overnight in YPD in triplicate with shaking. After 16 hours growth, liquid cultures were back-diluted to final OD₆₀₀=0.2 in 100 μL fresh YPD and pipetted into the wells of two 96 well plates (duplicates). One of the two plates was wrapped in black aluminum foil as a dark control. Both plates were placed under a LED lamp at 30° C. for 4 hours. Green fluorescence was then measured with a plate reader.

Light-inducible luciferase assay: Yeast strains were grown overnight in YPD in triplicate with shaking. After 16 hours growth, liquid cultures were back-diluted to final OD₆₀₀=0.2 in 15 μL fresh YPS and pipetted into the wells of two 96 well plates (duplicates for light and dark conditions, as previously described). Plates were placed under a LED lamp at 30° C. for 4 hours. Substrate in buffer from Nano-Glo® Luciferase Assay System (N1120, Promega) were added to the culture at 1:1 ratio at the end of incubation. After incubation in dark for 5 minutes, bioluminescence of the samples was measured with a plate reader.

Light-inducible pellicle response assay: Co-cultures were set up using yeast strains BY4741, yNC, and yNS along with wildtype K. rhaeticus in 10 mL YPS+OptiPrep. For long term exposure experiment, 60 mm petri dishes were prepared as duplicates, one was wrapped in black aluminum foil while the other one was not. The plates were placed under a LED lamp at 30° C. for 3 days. After the incubation, pellicles were flipped so the bottom side was facing up, and transferred onto YPD agar plates. 500 μL of Nano-Glo mix was applied onto the pellicles evenly through the entire surface. After incubation in dark for 10 minutes, bioluminescence of the samples was detected with a ChemiDoc Touch imager (BioRad). For short term exposure experiment (masking), co-cultures were grown in dark at 30° C. for 3 days. Pellicles were flipped so the bottom side was facing up, and transferred onto YPD agar plates. A mask made of black aluminum foil with carved pattern in the center was placed on top of the pellicles. Plates were placed under a LED lamp and incubated at 30° C. for 4 hours. Mask was then removed and 500 μL of Nano-Glo mix was applied onto the pellicles evenly through the entire surface. After incubation in dark for 10 minutes, bioluminescence of the samples was detected with a ChemiDoc Touch imager.

Light-patterning on pellicles: Co-cultures were grown in 100 mm square plates protected from light as previously described. Pellicles were rinsed in PBS, flipped, placed on YPD agar, and placed in an incubator with a projector mounted on top. After incubation under the projected pattern (with no lid to prevent water condensation) at 30° C. for 12 hours or more, 3 mL of Nano-Glo mix was applied onto the pellicles evenly through the entire surface. Bioluminescence images was detected with a ChemiDoc Touch imager after 30 minutes incubation in dark.

### REFERENCES

(1) Chen, A. Y., Zhong, C., and Lu, T. K. (2015) Engineering living functional materials. ACS Synth. Biol. 4, 8-11.
(2) Nguyen, P. Q. (2017) Synthetic biology engineering of biofilms as nanomaterials factories. Biochem. Soc. Trans. 45, 585-597.
(3) Nguyen, P. Q., Courchesne, N. D., Duraj-thatte, A., Praveschotinunt, P., and Joshi, N. S. (2018) Engineered Living Materials: Prospects and Challenges for Using Biological Systems to Direct the Assembly of Smart Materials. Adv. Mater. 1704847, 1-34.
(4) Gilbert, C., and Ellis, T. (2019) Biological Engineered Living Materials: Growing Functional Materials with Genetically Programmable Properties. ACS Synth. Biol. 8, 1-15.
(5) Blanco, L. P., Evans, M. L., Smith, D. R., Badtke, M. P., and Chapman, M. R. (2012) Diversity, biogenesis and function of microbial amyloids. Trends Microbiol. 20, 66-73.
(6) Kalyoncu, E., Ahan, R. E., Olmez, T. T., and Safak Seker, U. O. (2017) Genetically encoded conductive protein nanofibers secreted by engineered cells. RSC Adv. 7, 32543-32551.
(7) Seker, U. O. S., Chen, A. Y., Citorik, R. J., and Lu, T. K. (2017) Synthetic Biogenesis of Bacterial Amyloid Nanomaterials with Tunable Inorganic-Organic Interfaces and Electrical Conductivity. ACS Synth. Biol. 6, 266-275.
(8) Dorval Courchesne, N.-M., DeBenedictis, E. P., Tresback, J., Kim, J. J., Duraj-Thatte, A., Zanuy, D., Keten, S., and Joshi, N. S. (2018) Biomimetic engineering of conductive curli protein film. Nanotechnology 29, 509501.
(9) Chen, A. Y., Deng, Z., Billings, A. N., Seker, U. O. S., Lu, M. Y., Citorik, R. J., Zakeri, B., and Lu, T. K. (2014) Synthesis and patterning of tunable multiscale materials with engineered cells. Nat. Mater. 13, 515-23.
(10) Nguyen, P. Q., Botyanszki, Z., Tay, P. K. R., and Joshi, N. S. (2014) Programmable biofilm-based materials from engineered curli nanofibres. Nat. Commun. 5, 4945.
(11) Botyanszki, Z., Tay, P. K. R., Nguyen, P. Q., Nussbaumer, M. G., and Joshi, N. S. (2015) Engineered catalytic biofilms: Site-specific enzyme immobilization onto E. coli curli nanofibers. Biotechnol. Bioeng. 110, 2016-2024.
(12) Nussbaumer, M. G., Nguyen, P. Q., Tay, P. K. R., Naydich, A., Hysi, E., Botyanszki, Z., and Joshi, N. S. (2017) Bootstrapped biocatalysis: biofilm-derived materials as reversibly functionalizable multi-enzyme surfaces. ChemCatChem 9, 4328-4333.
(13) Tay, P. K. R., Nguyen, P. Q., and Joshi, N. S. (2017) A Synthetic Circuit for Mercury Bioremediation Using Self-Assembling Functional Amyloids. ACS Synth. Biol. 6, 1841-1850.
(14) Dorval Courchesne, N.-M., Duraj-Thatte, A., Tay, P. K. R., Nguyen, P. Q., and Joshi, N. S. (2016) Scalable Production of Genetically Engineered Nanofibrous Macroscopic Materials via Filtration. ACS Biomater. Sci. Eng. acsbiomaterials.6b00437.
(15) Wu, R. Q., Li, Z. X., Yang, J. P., Xing, X. H., Shao, D. Y., and Xing, K. L. (2010) Mutagenesis induced by high hydrostatic pressure treatment: A useful method to improve the bacterial cellulose yield of a Gluconoacetobacter xylinus strain. Cellulose 17, 399-405.
(16) Kuo, C. H., Teng, H. Y., and Lee, C. K. (2015) Knock-out of glucose dehydrogenase gene in Gluconacetobacter xylinus for bacterial cellulose production enhancement. Biotechnol. Bioprocess Eng. 20, 18-25.
(17) Yadav, V., Paniliatis, B. J., Shi, H., Lee, K., Cebe, P., and Kaplan, D. L. (2010) Novel in vivo-degradable cellulose-chitin copolymer from metabolically engineered Gluconacetobacter xylinus. Appl. Environ. Microbiol. 76, 6257-6265.
(18) Fang, J., Kawano, S., Tajima, K., and Kondo, T. (2015) In Vivo Curdlan/Cellulose Bionanocomposite Synthesis by Genetically Modified Gluconacetobacter xylinus. Biomacromolecules 16, 3154-3160.
(19) Florea, M., Hagemann, H., Santosa, G., Abbott, J., Micklem, C. N., Spencer-Milnes, X., de Arroyo Garcia, L., Paschou, D., Lazenbatt, C., Kong, D., Chughtai, H., Jensen, K., Freemont, P. S., Kitney, R., Reeve, B., and Ellis, T. (2016) Engineering control of bacterial cellulose production using a genetic toolkit and a new cellulose-producing strain. Proc. Natl. Acad. Sci. 201522985.
(20) Qin, G., Panilaitis, B. J., and Kaplan, Z. S. D. L. (2014) A cellulosic responsive “living” membrane. Carbohydr. Polym. 100, 40-45.
(21) Drachuk, I., Harbaugh, S., Geryak, R., Kaplan, D. L., Tsukruk, V. V., and Kelley-Loughnane, N. (2017) Immobilization of Recombinant E. coli Cells in a Bacterial Cellulose-Silk Composite Matrix to Preserve Biological Function. ACS Biomater. Sci. Eng. 3, 2278-2292.
(22) May, A. N., Medina, J., Alcock, J., Maley, C., and Aktipis, A. (2017) Kombucha as a model system for multispecies microbial cooperation: theoretical promise, methodological challenges and new solutions “in solution.” bioRxiv 214478.
(23) Jayabalan, R., Malini, K., Sathishkumar, M., Swaminathan, K., and Yun, S. E. (2010) Biochemical characteristics of tea fungus produced during kombucha fermentation. Food Sci. Biotechnol. 19, 843-847.
(24) Chen, C., and Liu, B. Y. (2000) Changes in major components of tea fungus metabolites during prolonged fermentation. J. Appl. Microbiol. 89, 834-839.
(25) Dufresne, C., and Farnworth, E. (2000) Tea, Kombucha, and health: A review. Food Res. Int. 33, 409-421.
(26) Baldwin, W., and Kubitschek, H. E. (1984) Buoyant Density Variation During the Cell Cycle of Saccharomyces cerevisiae. J. Bacteriol. 158, 701-704.
(27) Lee, M. E., DeLoache, W. C., Cervantes, B., and Dueber, J. E. (2015) A Highly-characterized Yeast Toolkit for Modular, Multi-part Assembly. ACS Synth. Biol. 4, 975-986.
(28) Ong, E., Gilkes, N. R., Miller, R. C., Warren, R. a, and Kilburn, D. G. (1993) The cellulose-binding domain (CBD(Cex)) of an exoglucanase from Cellulomonas fimi: production in Escherichia coli and characterization of the polypeptide. Biotechnol. Bioeng. 42, 401-9.
(29) Antošová, Z., Herkommerová, K., Pichová, I., and Sychrová, H. (2018) Efficient secretion of three fungal laccases from Saccharomyces cerevisiae and their potential for decolorization of textile industry effluent-A comparative study. Biotechnol. Prog. 34, 69-80.
(30) Viswanath, B., Rajesh, B., Janardhan, A., Kumar, A. P., and Narasimha, G. (2014) Fungal laccases and their applications in bioremediation. Enzyme Res. 2014.
(31) Li, J., Xu, H., Bentley, W. E., and Rao, G. (2002) Impediments to secretion of green fluorescent protein and its fusion from Saccharomyces cerevisiae. Biotechnol. Prog. 18, 831-838.
(32) Huang, D., and Shusta, E. V. (2005) Secretion and surface display of green fluorescent protein using the yeast Saccharomyces cerevisiae. Biotechnol. Prog. 21, 349-357.
(33) Harju, M., Kallioinen, H., and Tossavainen, O. (2012) Lactose hydrolysis and other conversions in dairy products: Technological aspects. Int. Dairy J. 22, 104-109.
(34) Bhosale, S. H., Rao, M. B., and Deshpande, V. V. (1996) Molecular and industrial aspects of glucose isomerase. Microbiol. Rev. 60, 280-300.
(35) DiCosimo, R., McAuliffe, J., Poulose, A. J., and Bohlmann, G. (2013) Industrial use of immobilized enzymes. Chem. Soc. Rev. 42, 6437.
(36) Shah, N., Ul-Islam, M., Khattak, W. A., and Park, J. K. (2013) Overview of bacterial cellulose composites: A multipurpose advanced material. Carbohydr. Polym. 98, 1585-1598.
(37) Wu, S.-C., and Lia, Y.-K. (2008) Application of bacterial cellulose pellets in enzyme immobilization. J. Mol. Catal. B Enzym. 54, 103-108.
(38) Wu, S.-C., Wu, S.-M., and Su, F.-M. (2017) Novel process for immobilizing an enzyme on a bacterial cellulose membrane through repeated absorption. J. Chem. Technol. Biotechnol. 92, 109-114.
(39) Allen, H. K., Donato, J., Wang, H. H., Cloud-Hansen, K. A., Davies, J., and Handelsman, J. (2010) Call of the wild: antibiotic resistance genes in natural environments. Nat. Rev. Microbiol. 8, 251-259.
(40) Crofts, T. S., Wang, B., Spivak, A., Gianoulis, T. A., Forsberg, K. J., Gibson, M. K., Johnsky, L. A., Broomall, S. M., Rosenzweig, C. N., Skowronski, E. W., Gibbons, H. S., Sommer, M. O. A., and Dantas, G. (2018) Shared strategies for β-lactam catabolism in the soil microbiome. Nat. Chem. Biol. 14, 556-564.
(41) Ostrov, N., Jimenez, M., Billerbeck, S., Brisbois, J., Matragrano, J., Ager, A., and Cornish, V. W. (2017) A modular yeast biosensor for low-cost point-of-care pathogen detection. Sci. Adv. 3, e1603221.
(42) Jarque, S., Bittner, M., Blaha, L., and Hilscherova, K. (2016) Yeast Biosensors for Detection of Environmental Pollutants: Current State and Limitations. Trends Biotechnol. 34, 408-419.
(43) Adeniran, A., Sherer, M., and Tyo, K. E. J. (2015) Yeast-based biosensors: Design and applications. FEMS Yeast Res. 15, 1-15.
(44) Adeniran, A., Stainbrook, S., Bostick, J., and Tyo, K. (2018) Detection of a peptide biomarker by engineered yeast receptors. ACS Synth. Biol. acssynbio.7b00410.
(45) McIsaac, R. S., Gibney, P. A., Chandran, S. S., Benjamin, K. R., and Botstein, D. (2014) Synthetic biology tools for programming gene expression without nutritional perturbations in Saccharomyces cerevisiae. Nucleic Acids Res. 42, 1-8.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. An isolated bacterial cellulose (BC)-based living composition, said BC-based living composition comprising a stable co-culture of at least one bacterial cellulose (BC)-producing bacteria strain and at least one synthetic biology host organism, wherein the synthetic biology host organism has been engineered to secrete one or more enzymes into bacterial cellulose (BC), and wherein the interaction between the bacteria strain and the synthetic biology host organism in the co-culture produces a self-assembled BC-based living composition.

2. The isolated BC-based living composition of claim 1, wherein the bacteria comprises Komagataeibacter rhaeticus (K. rhaeticus).

3. The isolated BC-based living composition of claim 1 or claim 2, wherein the synthetic biology host organism comprises an engineered yeast strain.

4. The isolated BC-based living composition of claim 3, wherein the engineered yeast strain comprises an engineered Saccharomyces cerevisiae (S. cerevisiae) strain.

5. The isolated BC-based living composition of any one of claims 1-4, wherein the BC-based living composition comprises a pellicle.

6. The isolated BC-based living composition of claim 5, wherein the pellicle comprises the BC-producing bacteria strain, the synthetic biology host organism, or both the BC-producing bacteria strain and the synthetic biology host organism.

7. The isolated BC-based living composition of any one of claims 1-6, wherein each of the one or more enzymes is linked to a cellulose binding protein or cellulose binding domain.

8. The isolated BC-based living composition of claim 7, wherein the cellulose binding protein or cellulose binding domain is CBDcex.

9. The isolated BC-based living composition of any one of claims 1-8, wherein each of the one or more enzymes is linked to a secretion signal peptide.

10. The isolated BC-based living composition of any one of claims 1-9, wherein expression of the one or more enzymes is controlled by a genetic circuit that responds to biological, chemical, or physical stimuli.

11. An isolated bacterial cellulose (BC)-based living composition, said composition comprising a stable co-culture of Komagataeibacter rhaeticus (K. rhaeticus) and an engineered Saccharomyces cerevisiae (S. cerevisiae) strain, wherein the engineered strain of S. cerevisiae has been engineered to secrete one or more enzymes into bacterial cellulose (BC), and wherein the interaction between the K. rhaeticus and the engineered S. cerevisiae in the co-culture produces a self-assembled BC-based living composition.

12. The isolated BC-based living composition of claim 11, wherein the BC-based living composition comprises a pellicle.

13. The isolated BC-based living composition of claim 12, wherein the pellicle comprises K. rhaeticus, the engineered S. cerevisiae strain, or both K. rhaeticus and the engineered S. cerevisiae strain.

14. The isolated BC-based living composition of any one of claims 11-13, wherein each of the one or more enzymes is linked to a cellulose binding protein or cellulose binding domain.

15. The isolated BC-based living composition of claim 12, wherein the cellulose binding protein or cellulose binding domain is CBDcex.

16. The isolated BC-based living composition of any one of claims 11-15, wherein each of the one or more enzymes is linked to a secretion signal peptide.

17. The isolated BC-based living composition of any one of claims 11-16, wherein expression of the one or more enzymes is controlled by a genetic circuit that responds to biological, chemical, or physical stimuli.

18. An isolated engineered strain of Saccharomyces cerevisiae (S. cerevisiae), wherein the engineered strain of S. cerevisiae secretes one or more enzymes into bacterial cellulose (BC).

19. The isolated engineered strain of S. cerevisiae of claim 18, wherein each of the one or more enzymes is linked to a cellulose binding protein or cellulose binding domain.

20. The isolated engineered strain of S. cerevisiae of claim 19, wherein the cellulose binding protein or cellulose binding domain is CBDcex.

21. The isolated engineered strain of S. cerevisiae of any one of claims 18-20, wherein each of the one or more enzymes is linked to a secretion signal peptide.

22. The isolated engineered strain of S. cerevisiae of any one of claims 18-21, wherein expression of the one or more enzymes is controlled by a genetic circuit that responds to biological, chemical, or physical stimuli.

23. An isolated co-culture of Komagataeibacter rhaeticus (K. rhaeticus) and an engineered strain of Saccharomyces cerevisiae (S. cerevisiae), wherein the engineered strain of S. cerevisiae secretes one or more enzymes into bacterial cellulose (BC).

24. The isolated co-culture of claim 23, wherein each of the one or more enzymes is linked to a cellulose binding protein or cellulose binding domain.

25. The isolated co-culture of claim 24, wherein the cellulose binding protein or cellulose binding domain is CBDcex.

26. The isolated co-culture of any one of claims 23-25, wherein each of the one or more enzymes is linked to a secretion signal peptide.

27. The isolated co-culture of any one of claims 23-26, wherein expression of the one or more enzymes is controlled by a genetic circuit that responds to biological, chemical, or physical stimuli.

28. A method of producing a bacterial cellulose (BC)-based living composition, said method comprising creating a stable co-culture of at least one bacterial cellulose (BC)-producing bacteria strain and at least one synthetic biology host organism, wherein the synthetic biology host organism strain has been engineered to secrete one or more enzymes into bacterial cellulose (BC), and wherein the interaction between the bacteria strain and the synthetic biology host organism in the co-culture produces a self-assembled BC-based living composition.

29. The method of claim 28, wherein the synthetic biology host organism comprises an engineered yeast strain.

30. The method of claim 29, wherein the engineered yeast strain comprises an engineered S. cerevisiae strain.

31. The method of any one of claims 28-30, wherein the bacterial strain comprises K. rhaeticus.

32. The method of any one of claims 28-31, wherein each of the one or more enzymes is linked to a cellulose binding protein or cellulose binding domain.

33. The method of claim 32, wherein the cellulose binding protein or cellulose binding domain is CBDcex.

34. The method of any one of claims 28-33, wherein each of the one or more enzymes is linked to a secretion signal peptide.

35. The method of any one of claims 28-34, wherein expression of the one or more enzymes is controlled by a genetic circuit that responds to biological, chemical, or physical stimuli.

36. The method of any one of claims 28-35, wherein the bacteria strain and the synthetic biology host organism are co-cultured for at least about 3 days.

37. The method of any one of claims 28-36, wherein the bacteria strain and the synthetic biology host organism are co-cultured in a culture medium that has a higher density than the bacteria strain and the synthetic biology host organism.

38. The method of any one of claims 28-37, wherein the bacteria strain and the synthetic biology host organism are co-cultured in sucrose-containing media.

39. A method of producing a bacterial cellulose (BC)-based living composition, said method comprising creating a stable co-culture of Komagataeibacter rhaeticus (K. rhaeticus) and an engineered Saccharomyces cerevisiae (S. cerevisiae) strain, wherein the engineered strain of S. cerevisiae has been engineered to secrete one or more enzymes into bacterial cellulose (BC), and wherein the interaction between the K. rhaeticus and the engineered S. cerevisiae in the co-culture produces a self-assembled BC-based living composition.

40. The method of claim 39, wherein each of the one or more enzymes is linked to a cellulose binding protein or cellulose binding domain.

41. The method of claim 40, wherein the cellulose binding protein or cellulose binding domain is CBDcex.

42. The method of any one of claims 39-41, wherein each of the one or more enzymes is linked to a secretion signal peptide.

43. The method of any one of claims 39-42, wherein expression of the one or more enzymes is controlled by a genetic circuit that responds to biological, chemical, or physical stimuli.

44. The method of any one of claims 39-43, wherein the K. rhaeticus and the engineered S. cerevisiae are co-cultured for at least about 3 days.

45. The method of any one of claims 39-44, wherein the K. rhaeticus and the engineered S. cerevisiae are co-cultured in a culture medium that has a higher density than the K. rhaeticus and the engineered S. cerevisiae.

46. The method of any one of claims 39-45, wherein the bacteria strain and the synthetic biology host organism are co-cultured in sucrose-containing media.

47. The method of any one of claims 28-46, wherein the BC-based living composition comprises an engineered living material (ELM).

48. The method of any one of claims 28-46, wherein the BC-based living composition comprises a pellicle.

49. Use of the isolated bacterial cellulose (BC)-based living composition of any one of claims 1 to 17 as a biosensor.

50. Use of the isolated BC-based living composition of any one of claims 1 to 17 for detecting microbe-microbe interactions.

51. Use of the isolated BC-based living composition of any one of claims 1 to 17 in a method for the detection and/or degradation of an environmental pollutant.

52. The use of claim 51 for the detection and/or degradation of one or more β-lactam antibiotics, one or more estrogen hormones, or a combination thereof.

53. Use of the isolated BC-based living composition of any one of claims 1 to 17 for detecting one or more pathogens in a sample.

54. Use of the isolated BC-based living composition of any one of claims 1 to 17 for detecting one or more biomarkers in a sample.

55. Use of the isolated BC-based living composition of any one of claims 1 to 17 in a living test paper or living film.

56. The use of claim 55, wherein the living test paper or living film generates an image in response to one or more stimuli.

57. The use of claim 56, wherein the one or more stimuli is light.
